(19) United States
(12) Patent Application Publication
(10) Pub. No.: US 2022/0118101 A1
(43) Pub. Date: Apr. 21, 2022

(54) COMPOSITIONS AND METHODS FOR PREVENTING OR REDUCING INFLAMMATION BY INHIBITING CASPASE-9

(71) Applicant: THE TRUSTEES OF COLUMBIA UNIVERSITY IN THE CITY OF NEW YORK, New York, NY (US)

(72) Inventors: Carol M. TROY, Hastings-on-Hudson, NY (US); Maria I. AVRUTSKY, Bronx, NY (US)

(21) Appl. No.: 17/515,202

(22) Filed: Oct. 29, 2021

Related U.S. Application Data
(63) Continuation of application No. PCT/US2020/030228, filed on Apr. 28, 2020.
(60) Provisional application No. 62/840,234, filed on Apr. 29, 2019.

(51) Int. Cl. A61K 47/64; A61P 27/02; A61K 38/57

(52) U.S. Cl. CPC A61K 47/64; A61K 38/1709; A61K 38/57; A61P 27/02

(57) ABSTRACT

The present disclosure relates to a method of preventing or decreasing inflammation in a patient comprising administering to the patient in need thereof an effective amount of a caspase-9 signaling pathway inhibitor. The caspase-9 signaling pathway inhibitor may include a peptide caspase-9 inhibitor and/or may be conjugated to a cell-penetrating peptide. The present disclosure further includes pharmaceutical compositions including a caspase-9 signaling pathway inhibitor. The disclosure further relates to the use of such compositions in a method of treating inflammation of the retina associated with retinal vein occlusion, diabetic macular edema, retinal detachment, ocular trauma, retinitis pigmentosa, age-related macular degeneration, uveitis, a retinal degenerative disease, glaucoma, Multiple sclerosis, Behcets, Lupus, Systemic sarcoidosis, Central serous chorioretinopathy, Leber's Hereditary Optic Neuropathy, Leigh Syndrome, Stargardt, retinitis pigmentosa, Best disease, or birdshot retinopathy.

### RELATED APPLICATIONS

This application is a continuation of PCT/US2020/030228, filed Apr. 28, 2020, which claims priority to U.S. Provisional Patent Application No. 62/840,234, filed Apr. 29, 2019, both of which are incorporated by reference in their entirety.

### STATEMENT OF GOVERNMENT INTEREST

This invention was made with government support under grants NS081333, NS099920, and EY013933 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is “210271_409C1_SEQUENCE-LISTING_01-07-2022.txt”. The text file is 16.9 KB, was created on Oct. 29, 2021, and is being submitted electronically via EFS-Web.

### TECHNICAL FIELD

The present disclosure relates to compositions and methods for preventing or reducing inflammation by inhibiting caspase-9.

### BACKGROUND

The biological role of caspase-9 is known to be complex, sometimes involving the intrinsic cell death pathway, but at other times having non-death functions. In addition, the non-death functions of caspase-9 exhibit even more complexity, with a large degree of variation in effects by tissue. Caspase-9 promotes muscle and cardiomyocyte differentiation and proliferation, hematopoietic development, neuronal maturation and axonal pathfinding, and activation of pro-survival NF-kB signaling. Furthermore, effector caspases-3 and -7 can promote wound healing and tissue regeneration by stimulating prostaglandin E₂ secretion via the phoenix rising pathway.

Caspase-9 has not previously been considered an inflammatory caspase, and has not previously been suggested as a target for modulating inflammatory response. However, caspase-9 has been shown to be necessary for normal immune system development and maintenance (e.g., Lu, E. P., et al. (2014). Blood 124, 3887-3895; Sordet, O., et al. (2002). Blood 100, 4446-4453) and genetic deletion of caspase-9 from endothelial and hematopoietic cells (Tie2-Cre Casp9 KO) causes mice to be more resistant to viral infection (Rongvaux, A., et al. (2014). Cell 159, 1563-1577).

### SUMMARY

The present disclosure surprisingly establishes that the caspase-9 is an inflammatory caspase, and that inhibition of a caspase-9 signaling pathway can prevent or decrease inflammation.

According to a first aspect, a method of preventing or decreasing inflammation in a patient is described. The method includes administering to the patient in need thereof an effective amount of a caspase-9 signaling pathway inhibitor.

The method can further include the following details, which can be combined with one another in any combinations unless clearly mutually exclusive:

(i) the inflammation may include neuroinflammation, appendicitis, bronchitis, bursitis, colitis, cystitis, dermatitis, encephalitis, gingivitis, meningitis, myelitis, nephritis, neuritis, periodontitis, pharyngitis, phlebitis, prostatitis, pulmonitis, retinitis, rhinitis, sinusitis, tendonitis, tonsillitis, urethritis, vaginitis, vasculitis, arthritis, myositis, arteritis, hepatitis, diverticulitis, otitis, uveitis, conjunctivitis, or episcleritis;

(ii) the neuroinflammation may include inflammation of a retina or a brain tissue;

(iii) the caspase-9 signaling pathway inhibitor may include a peptide caspase-9 inhibitor, a caspase-7 inhibitor or an Apaf-1 inhibitor;

(iv) the peptide caspase-9 inhibitor may include XBIR3;

(v) the caspase-9 signaling pathway inhibitor may be conjugated to a cell-penetrating peptide;

(vi) the cell-penetrating peptide may be selected from the group consisting of Penetratin1, transportan, pIS1, Tat(48-60), pVEC, MAP, MTS, a polyarginine, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3;

(vii) the caspase-9 signaling pathway inhibitor may include XBIR3 conjugated to Penetratin 1;

(viii) the inflammation of the retina may be associated with retinal vein occlusion, diabetic macular edema, retinal detachment, ocular trauma, retinitis pigmentosa, age-related macular degeneration, uveitis, a retinal degenerative disease, glaucoma, Multiple sclerosis, Behcets, Lupus, Systemic sarcoidosis, Central serous chorioretinopathy, Leber's Hereditary Optic Neuropathy, Leigh Syndrome, Stargardt, retinitis pigmentosa, Best disease, or birdshot retinopathy;

(ix) the administering may be via injection, inhalation, or topical administration.

According to a second aspect, a pharmaceutical composition is described. The pharmaceutical composition includes an effective amount of a caspase-9 signaling pathway inhibitor and a pharmaceutically acceptable carrier, wherein the pharmaceutical composition is formulated for administration to a patient via injection, inhalation, or topical administration.

The pharmaceutical composition can further include the following details, which can be combined with the above method and with one another in any combinations unless clearly mutually exclusive:

(i) the caspase-9 signaling pathway inhibitor may include a peptide caspase-9 inhibitor, a caspase-7 inhibitor or an Apaf-1 inhibitor;

(ii) the peptide caspase-9 inhibitor may include XBIR3;

(iii) the caspase-9 signaling pathway inhibitor may be conjugated to a cell-penetrating peptide;

(iv) the cell-penetrating peptide may be selected from the group consisting of Penetratin1, transportan, pIS1, Tat(48-60), pVEC, MAP, MTS, a polyarginine, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3;

(v) the XBIR3 may be conjugated to Penetratin1;

(vi) the effective amount may decrease or prevent inflammation of the retina associated with retinal vein occlusion, diabetic macular edema, retinal detachment, ocular trauma, retinitis pigmentosa, age-related macular degeneration, uveitis, a retinal degenerative disease, glaucoma, Multiple sclerosis, Behcets, Lupus, Systemic sarcoidosis, Central serous chorioretinopathy, Leber's Hereditary Optic Neuropathy, Leigh Syndrome, Stargardt, retinitis pigmentosa, Best disease, or birdshot retinopathy.

The compositions described herein may be used in the methods for treating inflammation described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present embodiments and advantages thereof may be acquired by referring to the following description taken in conjunction with the accompanying drawings, which relate to embodiments of the present disclosure. Certain abbreviations used in these figures and the descriptions thereof are explained in further detail in the remainder of this specification.

FIG. 1A is a set of exemplary micrographs showing a retinal section with segmentation of retinal layers and vascular plexi indicated, as observed by immunostaining (left panel; isolectin and DAPI) and optical coherence tomography (OCT) live imaging (middle panel and right panel, upper image) in an uninjured adult mouse. An exemplary micrograph of fundus retinal imaging in an uninjured adult mouse is also shown in the right panel, lower image.

FIG. 1B is a set of exemplary micrographs showing OCT (upper images) and fundus retinal imaging (lower images) at 4 hr, 24 hr, 48 hr, and 8 days post-retinal vein occlusion (RVO). Retinal detachment is indicated by arrowheads on the OCT images, and retinal hemorrhages are indicated by arrows on the fundus images.

FIG. 1C is a graph reporting exemplary quantification of change in total retinal thickness and retinal detachment post-RVO as measured by OCT. 13 to 21 eyes were analyzed per time-point. Data are presented as mean±SEM.

FIG. 1D is a graph reporting exemplary quantification of change in thickness of individual retinal layers. 4 to 21 eyes were analyzed per time-point. Data are presented as mean±SEM.

FIG. 1E is a graph reporting exemplary quantification of retinal thickness in uninjured control eyes, sham laser treated eyes, and RVO eyes at 4 hr, 24 hr, 48 hr, and 8 days. Data are presented as mean±SEM.

FIG. 1F is a graph reporting exemplary data showing retinal swelling is correlated to fraction of veins occluded 24 hr post-RVO. Line shows linear regression of best fit. n=25.

FIG. 2A is a set of exemplary micrographs of retinal flatmounts immunostained for the cell death marker Ph2AX and endothelial marker CD31 in control eyes, and at 4 hr and 24 hr post-RVO (scale bar=20 μm).

FIG. 2B is a set of exemplary micrographs of retinal flatmounts immunostained for cl-caspase 9 and endothelial marker CD31 in control eyes, and at 4 hr and 24 hr post-RVO (scale bar=20 μm).

FIG. 2C is a set of exemplary micrographs of retinal sections from control eyes and 24 hr post-RVO, immunostained for cl-casp7, cl-casp3, isolectin and DAPI. Inset shows vascular and neuronal induction of cl-casp7 24 hr post-RVO.

FIG. 3A is an exemplary schematic showing protein domains of XIAP (endogenous inhibitor of apoptosis protein). The Bir3 domain XIAP is a specific inhibitor of caspase-9. An exemplary composition, Pen1-XBir3, is generated by crosslinking XBir3 with Penetratin-1, a cell-penetrating peptide. Example ribbon diagrams of polypeptide structure of crosslinked Pen1-XBir3 were generated using the program Mol* (D. Sehnal, A. S. Rose, J. Kovca, S. K. Burley, S. Velankar (2018) Mol*: Towards a common library and tools for web molecular graphics MolVA/EuroVis Proceedings. doi:10.2312/molva.20181103).

FIG. 3B is an exemplary Western blot showing detection of XBir3 in retinal lysates at 1 hr, 2 hr, and 24 hr following administration of Pen1-XBir3 eye drops in mice. n=3.

FIG. 3C is an exemplary Western blot of retina lysates from mice that received Pen1-XBir3 eye drops immediately following induction of RVO. Immunoprecipitation of XBir3 from retinal lysates shows XBir3 binding to cl-casp9. n=2.

FIG. 3D is an exemplary Western blot in retinal lysates following administration of Pen1-XBir3 eye drops in rabbits for 5 consecutive days (left panel). The right panel is a graph reporting exemplary quantification of relative Pen1-XBir3 uptake (Student's t-test two-tailed vehicle vs. Pen1-XBir3, **p<0.01). n=5.

FIG. 3E is an exemplary Western blot of rabbit plasma after administration of Pen1-XBir3 eye drops for 5 consecutive days. n=3

FIG. 3F is an exemplary Western blot of retinal lysates 24 hr post-RVO (upper panel). Lower panels are graphs reporting exemplary quantification of VEGF and Casp9 levels (ANOVA with Fisher's LSD, mean±SEM; ***p<0.001, **p<0.01, *p<.05). n=4.

FIG. 4A is a set of exemplary micrographs of retinal sections from control eyes and 24 hr post-RVO with or without treatment with Pen1XBir3 eye drops, immunostained for CD31, cl-casp9, casp7, and DAPI. (scale bar=25 μm). n=3-8.

FIG. 4B is a graph reporting exemplary quantification of cells expressing cl-casp9 (ANOVA with Fisher's LSD, mean±SEM).

FIG. 4C is a graph reporting exemplary quantification of endothelial cells expressing casp7 (ANOVA with Fisher's LSD, mean±SEM).

FIG. 4D is a graph reporting exemplary quantification of neurons expressing casp7 (ANOVA with Fisher's LSD, mean±SEM).

FIG. 4E is a set of exemplary micrographs of retinal sections from control eyes and 24 hr post-RVO, immunostained for isolectin, casp8, and DAPI. (scale bar=25 μm). n=3-7.

FIG. 4F is a graph reporting exemplary quantification of neurons expressing casp8 (ANOVA with Fisher's LSD, mean±SEM).

FIG. 5A is a graph reporting exemplary quantification of vein dilation following RVO and treatment with Pen1-XBIR or vehicle. n=11-16 eyes analyzed per time point for each treatment group; two tailed Welch's t-test; mean±SEM.

FIG. 5B is a set of exemplary images of OCT fluorescein angiography and fundus retinal imaging of eyes treated with Pen1 or Pen1-XBir3, either alone or following induction of RVO.

FIG. 5C is a graph reporting exemplary quantification of fluorescein leakage from eyes treated with Pen1 or Pen1-XBir3 following induction of RVO. Data are presented as median extravascular fluorescein signal relative to uninjured Pen1-treated eyes. Pen1 n=5, Pen1-XBir3 n=7. Two-tailed Welch's t-test; mean±SEM.

FIG. 5D is a set of graphs reporting exemplary quantification of average change in thickness in specific retina layers as determined by OCT. n=11-21 eyes analyzed per time point for each treatment group. Student's t test (two-tailed) Pen1 vs Pen1-XBir3; mean±SEM.

FIG. 6A is a set of OCT images and micrographs of mouse retinas at 24 hrs post-RVO. Hyperreflective foci (HRF) in retinal vitreous are indicated by stars and hyperreflective foci in the inner nuclear layer are indicate by arrowheads. Insets show digitally zoomed-in high magnification images. (ANOVA, Dunnett's multiple comparisons test vs RVO+vehicle group; ****p<0.0001).

FIG. 6B is a graph reporting exemplary quantification of the number of HRF in the vitreous at 24 hr post-RVO. (ANOVA, Dunnett's multiple comparisons test vs RVO⁺ vehicle group; ***p<0.001). FIG. 6C is a graph reporting exemplary quantification of the number of HRF in the retina at 24 hr post-RVO. (ANOVA with Fisher's LSD, mean±SEM).

FIG. 6D is a set of exemplary phase images of retinal sections at 24 hrs post-RVO stained for TUNEL-positive nuclei and DAPI. Scale bar=20 p.m. The right panel is a graph reporting exemplary quantification of TUNEL+ cells in retinal sections 24 hr post-RVO; ANOVA with Fisher's LSD, mean±SEM.

FIG. 6E is a set of exemplary scotopic focal electroretinogram (ERG) traces obtained 7 days post-RVO. (flash intensity=2.3 log(Cd/m²).

FIG. 6F is a graph reporting exemplary quantification of B wave amplitudes from the ERG traces obtained 7 days post-RVO. (ANOVA with Fisher's LSD, mean±SEM). n=15-16.

FIG. 6G is a graph reporting exemplary quantification of b/a wave ratio 7 days post-RVO (ANOVA with Fisher's LSD, mean±SEM). n=15-16.

FIG. 6H is an exemplary micrograph of retinal sections treated with mutant inactive Pen1-XBir3 24 hr post-RVO immunostained for CD45, isolectin and DAPI. Inset shows magnification of a CD45+ leukocyte. In the magnified inset images on the right: the upper inset image shows the CD45+ leukocyte labeled for CD45, isolectin and DAPI; the middle inset image shows the CD45+ leukocyte labeled for isolectin; the lower inset image shows the CD45+ leukocyte labeled for CD45. N=7, scale bar=25 μm

FIG. 6I are graphs reporting exemplary quantifications of CD45+ leukocytes and HRF in eyes treated with Pen1-XBir3 and mutant inactive Pen1-mutXBir3 following induction of RVO.

FIG. 6J is a set of exemplary micrographs of retinal sections from uninjured control eyes (n=4), and eyes treated with Pen1-mut-XBir3 (n=7) or Pen1-XBir3 (n=6) following induction of RVO. Tissues are stained to detect CD45, isolectin and DAPI. scale bar=25 μm

FIG. 6K is a set of graphs reporting exemplary quantifications of CD45+ cells in retinal tissues following induction of RVO (upper panels; ANOVA with Fisher's LSD, mean±SEM), linear correlation between CD45+ leukocytes and fraction of veins occluded in RVO (lower left panel), and linear correlation between CD45+ leukocytes and retinal thickness in RVO (lower right panel).

FIG. 7A is a set of exemplary images from a Casp9 WT mouse (upper panels) and an endothelial caspase-9 knockout mouse (Casp9 iECKO mouse; lower panels) 24 hrs, 48 hrs, and 8 days post-RVO. OCT images are shown in the top of each panel, and fundus retinal images are shown in the bottom of each panel. Asterisks (*) indicate occluded veins, and dashed lines indicate areas of disorganization of retinal inner layers.

FIG. 7B is a set of graphs reporting exemplary quantification of OCT measurements of average % change in thickness of ganglion cell layer, inner plexiform layer, inner nuclear layer, outer plexiform layer, outer nuclear layer, at 0-8 days post-RVO in Casp9 WT animals and Casp9 iECKO animals, as compared to uninjured control animals. Also shown in FIG. 7B are graphs reporting exemplary quantification of % change in OS-RPE distance at 0-8 days post-RVO in Casp9 WT animals and Casp9 iECKO animals, as compared to uninjured control animals, and % change in retinal thickness at 0-8 days post-RVO in Casp9 WT animals and Casp9 iECKO animals, as compared to uninjured control animals (n=7-22, Two-tailed Welch's t-test; mean±SEM.)

FIG. 7C is a graph reporting exemplary quantification of fluorescein leakage in female Casp9 WT and female Casp 9 iECKO mice at 24 hours post-RVO. Data are presented as median extravascular fluorescein signal relative to uninjured vehicle-treated eyes. (Casp9 WT n=7, Casp9 iECKO n=6, Two-tailed Welch's t-test; mean±SEM.)

FIG. 7D is a graph reporting exemplary quantification of hyperreflective foci in the inner nuclear layer 48 hr post-RVO in Casp9 WT and Casp 9 iECKO mice. (one-way ANOVA with Fisher's LSD; mean±SEM.)

FIG. 7E is a graph reporting exemplary quantification of B wave amplitude 7 days post-ERG in Casp9 WT and Casp 9 iECKO mice (flash intensity=2.3 log(Cd/m²). (one-way ANOVA with Fisher's LSD; mean±SEM).

FIG. 7F is a set of exemplary micrographs of human brain sections from control (n=2, upper panels) and ischemic stroke (n=2, lower panels) patients immunostained for cleaved caspase 9 (cl-casp9) alone (left panels) or cl-casp9, CD31, and DAPI (right panels). Scale bar is 20 μm.

FIG. 7G is a set of exemplary micrographs of human brain sections from control patients (upper panels) and stroke patients (lower panels) immunostained for D315 cl-casp9, and CD31 (left panels) or D315 cl-casp9, CD31 and DAPI (right panels). Scale bar is 20 μm.

FIG. 7H is an exemplary schematic of a proposed model of caspase-9 activity in ischemic injury. Without being limited to theory, in the proposed model, endothelial caspase-9 activates caspase-7 by proteolytic cleavage, inducing endothelial barrier breakdown, retinal inflammation, and neuronal injury. Without being limited to theory, Pen1-XBir3, for example administered as eye-drops, inhibits caspase-9 and blocks the downstream effects.

FIG. 8 is a set of graphs reporting exemplary data showing retinal swelling in individual retinal layers is correlated to the fraction of veins occluded at 24 hr post-RVO. In each graph, the line shows linear regression of best fit. n=25.

FIG. 9A is a set of exemplary micrographs of retinal sections stained at 24 hr post-RVO for TUNEL, cl-Casp9, Isolectin and DAPI. Arrowheads indicate cl-Casp9-positive cells, and arrows indicate cells that are both TUNEL-positive and cl-Casp9-positive (scale bar=25 μm) n=5-6.

FIG. 9B. is a set of graphs showing exemplary quantifications of TUNEL+ cells correlated with fraction of veins occluded 24 hrs post-RVO, number of TUNEL+ cells in neurons and vasculature in uninjured control eyes and 24 hrs post-RVO, and number of cells positive for both TUNEL and cl-Casp9 in uninjured control eyes and 24 hrs post-RVO (n=5-6); Two-tailed Welch's t-test; mean±SEM.

FIG. 10 is a graph reporting exemplary quantification of hyperreflective foci in the retina inner nuclear layer in uninjured control eyes, and at 24 hr post-RVO in occluded (RVO) eyes and eyes where all veins had reperfused (RVO-reperfused). (ANOVA, Dunnett's multiple comparisons test vs Control group; ****p<0.0001).

FIG. 11 is set of exemplary green fluorescent protein (GFP) fluorescence micrographs of retinal flatmounts from a Tomato-EGFP reporter mouse (left panel) and a Tomato-EGFP X Cdh5(PAC)-CreERT2 mouse (right panel) following tamoxifen induction of recombination.

FIG. 12 is a set of exemplary fluorescence micrographs of a retinal flatmount from a Tomato-EGFP X Cdh5(PAC)-CreERT2 mouse following tamoxifen induction of recombination. Left panels were stained for GFP. Middle panels were stained for the endothelial marker CD31. Right panels show a combined image (composite) of the left and middle panels. Upper panels show larger vessels, while lower panels show microvasculature. Scale bar is 29 μm.

FIG. 13 is an exemplary image of an electrophoresis gel showing PCR products used to genotype a Casp9 wild type (WT) mouse (Ms1) and a Casp9iECKO mouse (Ms2) before and after tamoxifen treatment.

FIG. 14 is an exemplary set of micrographs of retinal sections obtained 24 hours post-RVO from a Casp9 WT mouse (top panels) and a Casp9 iECKO mouse (bottom panels). The retinal sections are immunostained for cl-Casp9, caspase-7, isolectin and DAPI. Scale bar is 25 μm.

FIG. 15 is a pair of exemplary fluorescein angiography images of the retinas of a Casp9 WT mouse (left image) and a Casp9 iECKO mouse (right image).

FIG. 16 is a pair of graphs reporting exemplary quantification of vein diameter (left graph) or artery diameter (right graph) in fluorescein angiography images of Casp9 WT mice (n=6) and Casp9 iECKO (n=4) mice.

FIG. 17 is a set of reporting exemplary quantification of scotopic ERG b-wave amplitude (left graph), a-wave amplitude (middle graph), and oscillatory potential (OP) amplitude (right graph) in Casp9 WT (n=24) mice and Casp9iECKO (n=19) mice under dim (−0.7 log(Cd/m²)) and bright (2.3 log(Cd/m²)) flash stimulus.

FIG. 18 is a pair of exemplary images of retinal fundus, in which the full retinal fundus view is indicated via a solid circle and retinal illumination by 1.5 mm spot size centered around optic nerve is indicated by a dashed circle. Laser burn site is indicated with arrows.

FIG. 19 is an exemplary Western blot of retinal lysates from untreated wildtype mice (control), Pen1-XBir3-treated wildtype mice (Pen1-XBir3), untreated wildtype mice 24 hr after induction of retinal detachment (RD), and Pen1-XBir3-treated wildtype mice 24 hr after induction of retinal detachment (RD+Pen1-XBir3). The Western blot membrane was probed for mouse immunoglobulin (IgG) and ERK1/2 loading control. RD induces 5-fold increase of IgG, which is abrogated by treatment with Pen1-XBir3.

FIG. 20 is an exemplary Western blot of retinal lysates from untreated wildtype mice (control), untreated wildtype mice 24 hr after induction of retinal vein occlusion (RVO), and Pen1-XBir3-treated wildtype mice 24 hr after induction of retinal vein occlusion (RVO+Pen1-XBir3). The Western blot was probed for IgG and ERK1/2 loading control. Increase in IgG after RVO is abrogated by treatment with Pen1-XBir3.

FIG. 21 is a graph reporting exemplary quantification of IgG heavy chain from FIG. 19.

FIG. 22 is a graph reporting exemplary quantification of IgG light chain from FIG. 19.

FIG. 23 is a graph reporting exemplary quantification of IgG heavy chain analyzed by western blot of retinal lysates of endothelial caspase-9 knockout mice (Casp9iECKO) and caspase-9 wildtype littermate controls (Casp9 WT). Retinal lysates were collected from intact retinas, and 24 hr post-RVO.

FIG. 24 is a graph reporting exemplary quantification of IgG light chain from western blot of retinal lysates of endothelial caspase-9 knockout mice (Casp9iECKO) and caspase-9 wildtype littermate controls (Casp9 WT). Retinal lysates were collected from intact retinas, and 24 hr post-RVO.

FIG. 25 is a graph reporting exemplary data on anti-inflammatory/protective cytokines induced by caspase-9 inhibition.

FIG. 26 is a graph reporting exemplary data on cytokines reduced by caspase-9 inhibition.

FIG. 27 is a graph reporting exemplary data on effect of caspase-9 inhibition on VEGF signaling pathway.

### DETAILED DESCRIPTION

Previously, increased caspase-9 activity or expression has been linked to various pathological conditions. Typically, increased activity or expression of caspase-9 has previously been assumed to indicate apoptotic cell death. The present disclosure establishes, contrary to prior assumptions, that non-apoptotic caspase-9 activity may promote inflammation, and that blocking caspase-9 expression and/or activation may prevent or decrease inflammation. Accordingly, in various pathological conditions associated with increased caspase-9 expression and/or activity, it is contemplated that blocking caspase-9 expression and/or activation may prevent or decrease symptoms associated with inflammation.

As used herein, the term “inflammation” refers to part of a complex biological response of body tissues to harmful stimuli, such as pathogens, damaged cells, or irritants, among others, and may be, at least initially, a protective response, that may involve immune cells, blood vessels, and molecular mediators. In general, the function of inflammation is to eliminate the initial cause of cell injury, clear out necrotic cells and tissues damaged from the original insult and the inflammatory process, and initiate tissue repair. Symptoms of inflammation in a patient may include heat, pain, redness, swelling, and loss of function. Inflammation is generally considered to be a generic mechanism of innate immunity. This contrasts with adaptive immunity, which typically includes a specific immune response for a specific pathogen. In some cases, too little inflammation can lead to progressive tissue destruction by the harmful stimulus and compromise the survival of the patient. In contrast, chronic inflammation may lead to a host of diseases. Inflammation is therefore normally closely regulated by the body. Inflammation can be classified as either acute or chronic. Acute inflammation refers to the initial response of the body to harmful stimuli and involves increased movement of plasma and leukocytes from the blood into the injured tissues. A series of biochemical events propagates and matures the inflammatory response, involving the local vascular system, the immune system, and various cells within the injured tissue. Prolonged inflammation, referred to as chronic inflammation, leads to a progressive shift in the type of cells present at the site of inflammation, such as mononuclear cells, and may be characterized by simultaneous destruction and healing of the tissue from the inflammatory process.

Acute inflammation is typically a short-term process, usually appearing within a few minutes or hours and begins to cease upon the removal of the injurious stimulus. It involves a coordinated and systemic mobilization response locally of various immune, endocrine and neurological mediators of acute inflammation. In a normal healthy response, it becomes activated, clears the pathogen and begins a repair process and then ceases. The process of acute inflammation is typically initiated by resident immune cells already present in the involved tissue, such as resident macrophages, dendritic cells, histiocytes, Kupffer cells and mast cells. These cells possess surface receptors known as pattern recognition receptors (PRRs), which bind to and thereby recognize two subclasses of molecules: pathogen-associated molecular patterns (PAMPs) and damage-associated molecular patterns (DAMPs). PAMPs are compounds that are associated with various pathogens, but which are distinguishable from host molecules. DAMPs are compounds that are associated with host-related injury and cell damage. At the onset of an infection, burn, or other injuries, these cells undergo activation (e.g., one of the PRRs recognize a PAMP or DAMP) and release inflammatory mediators responsible for the clinical signs of inflammation. The mediator molecules also alter the blood vessels to permit the migration of leukocytes, mainly neutrophils and macrophages, outside of the blood vessels (referred to as extravasation) into the tissue.

In addition to cell-derived mediators, several biochemical cascade systems involving plasma proteins act in parallel to initiate and propagate the inflammatory response. These include without limitation, bradykinin, complement system proteins such as C3, C5a, C5b, C6, C7, C8 and C9, Factor XII, plasmin, and thrombin, among others. Acute inflammation can also involve the movement of plasma fluid containing proteins such as fibrin and immunoglobulins into inflamed tissue.

The cellular component of acute inflammation often involves leukocytes, which normally reside in blood and move into the inflamed tissue via extravasation to aid in inflammation. Some act as phagocytes, ingesting bacteria, viruses, and cellular debris. Others release enzymatic granules that damage pathogenic invaders. Leukocytes also release inflammatory mediators that develop and maintain the inflammatory response. In general, acute inflammation may be mediated by granulocytes, for example, whereas chronic inflammation may be mediated by mononuclear cells such as monocytes and lymphocytes. Cell derived-mediators of inflammation include, without limitation, lysosome granules, histamine, interferon-γ, interleukin-8, leukotriene B4, leukotriene C4, leukotriene D4, 5-oxo-eicosatetraenoic acid, 5-Hydroxyeicosatetraenoic acid, prostaglandins, nitric oxide, tumor necrosis factor alpha, interleukin-1, and tryptase, among others.

In some cases, specific patterns of acute and chronic inflammation are seen during particular situations that arise in the body. For example, granulomatous inflammation refers to the formation of granulomas, associated with diseases such as tuberculosis, leprosy, sarcoidosis, and syphilis. Fibrinous inflammation refers to inflammation resulting in a large increase in vascular permeability allows fibrin to pass through the blood vessels. If an appropriate pro-coagulative stimulus is present, such as cancer cells, a fibrinous exudate may be deposited. This is commonly seen in serous cavities, where the conversion of fibrinous exudate into a scar can occur between serous membranes, limiting their function. The deposit sometimes forms a pseudo-membrane sheet. During inflammation of the intestine (e.g., pseudomembranous colitis), pseudo-membranous tubes can be formed. Purulent inflammation refers to inflammation resulting in a large amount of pus, which may contain neutrophils, dead cells, and fluid. For example, infection by pyogenic bacteria such as staphylococci is characteristic of purulent inflammation. Large, localized collections of pus enclosed by surrounding tissues are referred to as abscesses. Serous inflammation refers to copious effusion of non-viscous serous fluid, commonly produced by mesothelial cells of serous membranes, but may be derived from blood plasma. Skin blisters exemplify serous inflammation. Ulcerative inflammation refers to inflammation occurring near an epithelium and can result in the necrotic loss of tissue from the surface, exposing lower layers. The subsequent excavation in the epithelium is known as an ulcer.

Inflammation can result from various causes, including, without limitation, physical causes such as burns, frostbite, blunt or penetrating injury, foreign bodies e.g. splinters, dirt and debris, trauma, ionizing radiation; biological causes such as infection by pathogens, immune reactions due to hypersensitivity, and stress; chemical causes such as chemical irritants, toxins, or alcohol, among other causes identifiable by skilled persons.

By convention, many types of inflammation are indicated by the suffix “-itis”, and are often associated with a particular tissue of a patient. For example, some types of inflammation include, without limitation, appendicitis, bronchitis, bursitis, colitis, cystitis, dermatitis, encephalitis, gingivitis, meningitis, myelitis, nephritis, neuritis, periodontitis, pharyngitis, phlebitis, prostatitis, pulmonitis, rhinitis, sinusitis, tendonitis, tonsillitis, urethritis, vaginitis, and vasculitis, among others described herein or identifiable by skilled persons upon reading the present disclosure.

The term “vasculitis” refers to a group of disorders that destroy blood vessels by inflammation. Arteries and/or veins may be affected in vasculitis. Without limitation to theory, vasculitis is often caused by leukocyte migration and resultant damage. Possible symptoms include, without limitation, general symptoms such as fever and weight loss, skin symptoms such as palpable purpura and livedo reticularis, muscle and joint symptoms such as myalgia, myositis, arthralgia and arthritis, nervous system symptoms such as mononeuritis multiplex, headache, stroke, tinnitus, reduced visual acuity, and acute visual loss, heart and artery symptoms such as myocardial infarction, hypertension, and gangrene, respiratory tract symptoms such as nose bleeds, bloody cough, and lung infiltrates gastrointestinal tract symptoms such as abdominal pain, bloody stool, and perforations, and kidney symptoms such as glomerulonephritis, among others. For example, vasculitis may include without limitation, cutaneous small-vessel vasculitis which may affect the skin and kidneys; granulomatosis with polyangiitis which may affect nose, lungs, and kidneys; eosinophilic granulomatosis with polyangiitis which may affect lungs, kidneys, heart, and skin; Behçet's disease which may affect sinuses, brain, eyes, skin, lungs, kidneys, and joints; Kawasaki disease which may affect skin, heart, mouth, and eyes; Buerger's disease which may affect leg arteries and veins; Takayasu's arteritis, polyarteritis nodosa and giant cell arteritis which may affect arteries. Some diseases have vasculitis as an accompanying feature, including, without limitation, rheumatic diseases, such as rheumatoid arthritis, systemic lupus erythematosus, and dermatomyositis; cancers, such as lymphomas; infections, such as hepatitis C. In addition, exposure to certain chemicals and drugs, such as amphetamines, cocaine, and anthrax vaccines which contain the Anthrax Protective Antigen as the primary ingredient may be associated with vasculitis. In pediatric patients, varicella inflammation may be followed by vasculitis of intracranial vessels. This condition is referred to as post varicella angiopathy and may be associated with arterial ischemic strokes in children. Vasculitis may be diagnosed in a patient using various methods known in the art. For example, laboratory tests of blood or body fluids may show signs of inflammation in the body, such as increased erythrocyte sedimentation rate (ESR), elevated C-reactive protein (CRP), anemia, increased white blood cell count and eosinophilia, among other signs. A biopsy of an affected organ or tissue, such as skin, sinuses, lung, nerve, brain and kidney, and so on, or an angiogram (x-ray assay of the blood vessels) may reveal the pattern of blood vessel inflammation. 18F-fluorodeoxyglucose positron emission tomography/computed tomography (FDG-PET/CT) imaging may be used, e.g. in patients with suspected Large Vessel Vasculitis, due to the enhanced glucose metabolism of inflamed vessel walls.

Endothelial inflammation can include inflammation of the cells that line the interior surface of blood vessels and lymphatic vessels. The endothelium refers to a thin layer of squamous cells called endothelial cells. Endothelial cells in direct contact with blood are called vascular endothelial cells, whereas those in direct contact with lymph are known as lymphatic endothelial cells.

Inflammation is associated with a large group of disorders that underlie a variety of diseases. The immune system is often involved with inflammatory disorders, for example allergic reactions and some myopathies, with many immune system disorders resulting in abnormal inflammation. Non-immune diseases with causal origins in inflammatory processes include, without limitation, cancer, atherosclerosis, and ischemic heart disease, among others described herein or identifiable by skilled persons upon reading of the present disclosure. Examples of disorders associated with inflammation include, without limitation, acne vulgaris, atherosclerosis, asthma, autoimmune diseases, autoinflammatory diseases, celiac disease, chronic prostatitis, colitis, diverticulitis, glomerulonephritis, hidradenitis suppurativa, Acquired Immune Deficiency Syndrome, hypersensitivities, inflammatory bowel diseases, interstitial cystitis, ischemia, lichen planus, Mast Cell Activation Syndrome, mastocytosis, otitis, pelvic inflammatory disease, reperfusion injury, rheumatic fever, rheumatoid arthritis, rhinitis, sarcoidosis, transplant rejection, and vasculitis, among others described herein or identifiable by skilled persons upon reading of the present disclosure. Allergic reactions, otherwise known as type 1 hypersensitivity results from an inappropriate immune response, triggering inflammation. A severe allergic inflammatory response may become a systemic response known as anaphylaxis. Inflammatory myopathies cause muscle inflammation and may occur in immune disorders such as systemic sclerosis, dermatomyositis, polymyositis, and inclusion body myositis.

In a normal inflammation response, the acute inflammatory response is typically terminated when no longer needed to prevent unnecessary “bystander” damage to tissues. Failure to terminate acute inflammation may result in chronic inflammation, and may include damage including without limitation cell death in the patient's tissues. When inflammation overwhelms the patient, systemic inflammatory may occur. In such cases, systemic inflammatory response syndrome may be diagnosed. For example, the term sepsis may refer to chronic inflammation associated with an infection. The term bacteremia refers to bacterial sepsis and viremia refers to viral sepsis. Vasodilation and organ dysfunction are serious problems that are often associated with widespread infection and inflammation that may lead to septic shock and death.

Inflammation may involve high systemic levels of acute-phase proteins. In acute inflammation, these proteins may be beneficial; however, in chronic inflammation they can contribute to amyloidosis. These proteins include, without limitation, C-reactive protein, serum amyloid A, and serum amyloid P, among others, which are associated with a range of systemic effects such as fever, increased blood pressure, decreased sweating, malaise, loss of appetite, and somnolence. Inflammation often affects the numbers of leukocytes present in the body of a patient. Leukocytosis often occurs during inflammation induced by infection, where it results in a large increase in the amount of leukocytes in the blood, especially immature cells. In leukocytosis, leukocyte numbers may increase to between, or between about, 15,000 and 20 000 cells per microliter, or up to about, 100 000 cells per microliter. Bacterial infection often results in an increase of neutrophils, creating neutrophilia, whereas diseases such as asthma, hay fever, and parasite infestation may result in an increase in eosinophils, creating eosinophilia.

Systemic inflammation is typically not confined to a particular tissue but may involve the endothelium and other organ systems. Increased levels of several markers of inflammation may be present, such as interleukin-6, interleukin-8, interleukin-18, tumor necrosis factor-alpha, and C-reactive protein, among others. Chronic inflammation may last days, months or even years, and may lead to the formation of a chronic wound. Chronic inflammation often involves increased presence of macrophages in the injured tissue, which may release toxins that are injurious to the patient's own tissues.

The present disclosure relates to compositions and methods for preventing or decreasing inflammation by inhibiting a caspase-9 signaling pathway. Without being limited to a particular mode of action, caspase-9 acts via a signaling pathway that does not involve modulation of VEGF-A levels, or induction of apoptosis in the cells expressing activated caspase-9.

In some embodiments, the effects may occur in one or more neuronal tissues, including tissues of the central nervous system or the peripheral nervous system such as the brain, spinal cord, nerves, and eye, such as in the retina of the eye. In some embodiments, the effects may occur in one or more other tissues, such as in the gut, blood, lymph, muscle, skeletal, skin, or other tissues described herein or identifiable by skilled persons upon reading the present disclosure.

As used herein, the term “patient” refers to any animal, including any mammal, including, but not limited to, humans, and non-human animals (including, but not limited to, non-human primates, dogs, cats, rodents, horses, cows, pigs, mice, rats, hamsters, rabbits, and the like. In particular, the patient is a human.

As used herein, an “effective amount” is an amount sufficient to cause a beneficial or desired clinical result in a patient. An effective amount can be administered to a patient in one or more doses. In terms of treatment, an effective amount is an amount that is sufficient to prevent or decrease inflammation in a patient, including without limitation decreasing inflammation in a tissue of a patient, or systemically in a patient. In some embodiments, the effect amount may also ameliorate neurodegeneration resulting from inflammation. The effective amount is generally determined by the physician on a case-by-case basis and is within the skill of one in the art. Several factors may be taken into account when determining an appropriate dosage to achieve an effective amount. These factors include age, the condition being treated, the severity of the condition, prior responses, type of inhibitor used, the caspase-9 signaling pathway member to be inhibited, the cell type expressing the target, and the form and effective concentration of the composition (also referred to herein as a “treatment,” “inhibitor,” or “conjugate”) being administered.

As used herein, “treat,” “treating” and similar verbs refer to ameliorating, preventing or reducing inflammation and optionally ameliorating, preventing or reducing neurodegeneration resulting from inflammation in a patient.

In some embodiments, the compositions and methods described herein can be used for inhibiting a caspase-9 signaling pathway and thereby decreasing inflammation associated with various pathological conditions. For example, the compositions and methods described herein may be used for reducing retinal inflammation in diseases having an inflammatory component. In some embodiments, the term “neuroinflammation” refers to inflammation of a nervous system tissue. For example, such diseases include without limitation RVO, diabetic macular edema, retinal detachment, ocular trauma, retinitis pigmentosa, age-related macular degeneration (AMD), uveitis, retinal degenerative diseases, and glaucoma, among others described herein or identifiable by skilled persons upon reading the present disclosure. Exemplary retinal diseases include, without limitation, Leber's Hereditary Optic Neuropathy, Leigh Syndrome, Stargardt, retinitis pigmentosa, Best disease, and birdshot retinopathy, among others. The compositions and methods described herein may also be used for reducing ocular inflammation in non-hereditary inflammatory conditions including, without limitation, dry eye disease (conjunctivitis), episcleritis, and atopic dermatitis, among others. In some embodiments, the compositions and methods described herein can be used for treating neuroinflammatory injury in central nervous system (CNS) tissues, such as neuroinflammatory injury in Multiple sclerosis, Behcets, Lupus, Systemic sarcoidosis, and Central serous chorioretinopathy, among others. In some embodiments, the compositions and methods described herein can be used for treating ocular pathology in systemic neuroinflammatory diseases, such as Behcet's disease, Multiple sclerosis, or systemic Lupus erythematosus, among others described herein or identifiable by skilled persons upon reading the present disclosure. In some embodiments, the compositions and methods described herein can be used for treating vasculitis. In some embodiments, the compositions and methods described herein can be used for reducing IgG leakage, for example to treat sepsis, attenuate cytokine storm, or modulate innate immune response, among others described herein or identifiable by skilled persons upon reading the present disclosure. In some embodiments, the compositions and methods described herein can be used for treating inflammatory conditions including without limitation inflammatory bowel disease (IBD), rhegmatogenous retinal detachment, ischemic stroke, amyotrophic lateral sclerosis (ALS), or atherosclerosis, among others described herein or identifiable by skilled persons upon reading the present disclosure. In some embodiments, the compositions and methods described herein can be used to treat inflammatory consequences of XIAP deficiency disorder or lymphoproliferative syndrome.

The Examples described herein demonstrate an anti-inflammatory effect of caspase-9 inhibition in two exemplary models of retinal injury, a retinal vein occlusion (RVO) model and a retinal detachment (RD) model (see, e.g. Examples 11, 12, 15, 16, 18, 19 and 20).

The anti-inflammatory effect of caspase-9 inhibition is demonstrated using two exemplary approaches: (1) pharmacological inhibition via Pen-XBir3 eye drops, and (2) genetic deletion of caspase-9 specifically in endothelial cells (Casp9iECKO mice). The Examples show that blocking caspase-9 by administration of Pen-XBir3 eye drops reduces immunoglobulin (IgG) levels in retina in RD and RVO models of retinal injury, and that Casp9iECKO mice have lower levels of IgG after RVO.

The Examples also show that blocking caspase-9 reduces the number of retinal hyperreflective foci (HRF) after RVO. In addition, Casp9iECKO mice have fewer HRF after RVO. Hyperreflective foci are not specific to hypoxia or ischemia injury. Instead, HRF are a nonspecific indicator of retinal inflammation and are present, for example, in diseases which do not feature hypoxia or ischemia, such as Stargardt, uveitis, Best vitelliform macular dystrophy, retinitis pigmentosa, birdshot retinopathy, among others. Accordingly, in some embodiments, the methods and compositions of the present disclosure are suitable for example, without limitation, for treating diseases that have an inflammatory component, and that do not feature hypoxia or ischemia.

### Methods of Preventing or Decreasing Inflammation and Preventing or Decreasing Neurodegeneration

In certain embodiments, the instant disclosure is directed to methods of or uses of treatments disclosed herein in preventing or reducing inflammation and optionally also preventing or reducing neurodegeneration resulting from inflammation in a patient by administering an effective amount of a caspase-9 signaling pathway inhibitor. In some embodiments, the caspase-9 signaling pathway inhibitor may be a caspase-9 inhibitor, a caspase-7 inhibitor, or an Apaf-1 inhibitor. In some embodiments, the caspase-9 signaling pathway inhibitor may be a cell-penetrating caspase-9 signaling pathway inhibitor. In certain embodiments, the methods of the present disclosure are directed to the administration of the cell-penetrating caspase-9 signaling pathway inhibitor via injection, inhalation, or topical administration. Injection may be intravenous, intraocular, intraarterial, intracerebral, intracerebroventricular, or sub-tenon's injection. Topical administration may be via eye drops, or directly onto skin, or nasal spray.

The treatment may be administered as a single dose or multiple doses. For example, but not by way of limitation, where multiple doses are administered, they may be administered at intervals of 6 times per 24 hours or 4 times per 24 hours or 3 times per 24 hours or 2 times per 24 hours or 1 time per 24 hours or 1 time every other day or 1 time every 3 days or 1 time every 4 days or 1 time per week, or 2 times per week, or 3 times per week. In certain embodiments, the initial dose may be greater than subsequent doses or all doses may be the same.

In certain embodiments, the cell-penetrating caspase-9 signaling pathway inhibitor used in connection with the methods and uses of the instant disclosure is a Pen1-XBIR3 conjugate or a Pen1-XBIR2 conjugate as disclosed herein. The concentration of the Pen1-XBIR3 composition or the Pen1-XBIR2 composition administered is, in certain embodiments: 0.1 μM to 1,000 μM; 1 μM to 500 μM; 10 μM to 100 μM; or 20 μM to 60 μM. In certain embodiments, a specific human equivalent dosage can be calculated from animal studies via body surface area comparisons, as outlined in Reagan-Shaw et al., FASEB J., 22; 659-661 (2007).

In certain embodiments, the caspase-9 signaling pathway inhibitor, either alone or in the context of a membrane-permeable conjugate, is administered in conjunction with one or more additional therapeutics. In certain embodiments the method involves the administration of one or more additional caspase-9 signaling pathway inhibitors either alone or in the context of a membrane-permeable conjugate.

### Compositions

Caspase-9 Signaling Pathway Inhibitors

In certain embodiments, the caspase-9 signaling pathway inhibitors of the present disclosure may be peptide inhibitors of caspase-9 or peptide inhibitors of caspase-7.

In certain embodiments, the peptide inhibitors of caspase-9 or the peptide inhibitors of caspase-7 include, but are not limited to the class of protein inhibitors identified as Inhibitors of Apoptosis (“IAPs”). IAPs generally contain one to three BIR (baculovirus IAP repeats) domains, each consisting of approximately 70 amino acid residues. In addition, certain IAPB also have a RING finger domain, defined by seven cysteines and one histidine (C₃HC₄) that can coordinate two zinc atoms.

Exemplary mammalian IAPB suitable for use herein, include, but are not limited to c-IAP1 (e.g., Accession No. Q13490.2), cIAP2 (e.g., Accession No. Q13489.2), and XIAP (e.g., Accession No. P98170.2), each of which have three BIRs in the N-terminal portion of the molecule and a RING finger at the C-terminus. NAIP (e.g., Accession No. Q13075.3), another suitable mammalian IAP, contains three BIRs without RING, and survivin (e.g., Accession No. 015392.2) and BRUCE (e.g., Accession No. Q9H8B7), which are two additional suitable IAPB, both of which contain just one BIR.

In certain embodiments, the peptide inhibitor of caspase-9 is the third BIR domain of XIAP, referred to herein as “XBIR3”. For example, in some embodiments, the XBIR3 has the sequence

(SEQ ID NO: 14)
STNLPRNPSMADYEARIFTFGTWIYSVNKEQLARAGFYALGEGDKVKCF
HCGGGLTDWKPSEDPWEQHAKWYPGCKYLLEQKGQEYINNIHLTHS.

In certain embodiments, the peptide inhibitor of caspase-9 is XBIR3 having the sequence

MGSSHHHHHHSSGLVPRGSHMSTNTCLPRNPSMADYEARIFTFGTWIYS VNKEQLARAGFYTDWALGEGDKVKCFHCGGGLRPSEDPWEQHARWYPGCRY LLEQRGQEYINNIHLTHS (SEQ ID NO: 1). The amino acid sequence of SEQ ID NO:1 includes a His-tag near the N-terminus having the sequence HHHHHH (SEQ ID NO:15). In certain embodiments, the peptide inhibitors of caspase-9 such as that of SEQ ID NO:1, and other described herein, do not include a His-tag. For example, in some embodiments where the peptide inhibitor of caspase-9 is intended to be administered to a patient, the peptide inhibitor of caspase-9 may lack a His-tag.

In certain embodiments the peptide inhibitor of caspase-9 is XBIR3 having the sequence

MGSSHHHHHHSSGLVPRGSHMSTNTLPRNPSMADYEARIFTFGTWIYSV NKEQLARAGFYTDWALGEGDKVKCFHCGGGLRPSEDPWEQHARWYPGCRYLL EQRGQEYINNIHLTHS (SEQ ID NO: 2). The amino acid sequence of SEQ ID NO:2 includes a His-tag near the N-terminus having the sequence HHHHHH (SEQ ID NO:15). In certain embodiments, the peptide inhibitors of caspase-9 such as that of SEQ ID NO:2, and other described herein, do not include a His-tag. For example, in some embodiments where the peptide inhibitor of caspase-9 is intended to be administered to a patient, the peptide inhibitor of caspase-9 may lack a His-tag.

In certain embodiments the peptide inhibitors of caspase-9 include, but are not limited to Z-LEHD-AMC (WO 2006056487); z-LEHD-fmk, Z-VAD-FMK, CrmA, and Z-VAD-(2, 6-dichlorobenzoyloxopentanoic acid) (Garcia-Calvo, et al., J. Biol. Chem., 273, 32608-32613 (1998)) among others described herein or identifiable by skilled persons upon reading the present disclosure.

In certain embodiments, inhibitors of caspase-9 include small molecule inhibitors of apoptotic protease activating factor-1 (Apaf-1), such as compounds Leonurine (also known as SCM-198), ZYZ-488, or QM31 (also known as SVT016426). Leonurine is a natural alkaloid that may occupy the caspase recruitment site of Apaf-1, blocking its interaction with procaspase-9. ZYZ-488 is an inhibitor of Apaf-1 that may inhibit the activation of procaspase-9 and procaspase-3. QM31 may inhibit the formation of the apoptosome, the caspase activation complex composed of Apaf-1, cytochrome c, dATP and caspase-9.

In certain embodiments, the peptide inhibitor of caspase-7 is the second BIR domain of XIAP, referred to herein as “XBIR2”. For example, in some embodiments, the XBIR2 has the sequence EEARLKSFQNWPDYAHLTPRELASAGLYYTGIGDQVQCFCCGGKLKNW EPCDRAWSEHRRHFPNCFFV (SEQ ID NO: 16), corresponding to residues 163-230 of human XIAP (e.g. Accession No. P98170.2). Peptide inhibitors of caspase-9 or caspase-7 include those amino acid sequences that retain certain structural and functional features of the identified caspase-9 inhibitor or caspase-7 inhibitor peptides, yet differ from the identified inhibitors' amino acid sequences at one or more positions. Such variants can be prepared by substituting, deleting, or adding amino acid residues from the original sequences via methods known in the art.

In certain embodiments, such substantially similar sequences include sequences that incorporate conservative amino acid substitutions. As used herein, a “conservative amino acid substitution” is intended to include a substitution in which the amino acid residue is replaced with an amino acid residue having a similar side chain. Families of amino acid residues having similar side chains have been defined in the art, including: basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine); nonpolar side chains (e.g., alanine, valine, leucine, isoleucine, proline, phenylalanine, methionine, tryptophan); (β-branched side chains (e.g., threonine, valine, isoleucine); and aromatic side chains (e.g., tyrosine, phenylalanine, tryptophan, histidine). Other generally preferred substitutions involve replacement of an amino acid residue with another residue having a small side chain, such as alanine or glycine. Amino acid substituted peptides can be prepared by standard techniques, such as automated chemical synthesis.

In certain embodiments, a peptide inhibitor of caspase-9 or caspase-7 of the present disclosure is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the original peptide inhibitor of caspase-9 or caspase-7, such as an IAP, and is capable of caspase-9 or caspase-7 inhibition. As used herein, the percent homology between two amino acid sequences may be determined using standard software such as BLAST or FASTA. The effect of the amino acid substitutions on the ability of the synthesized peptide inhibitor of caspase-9 or caspase-7 to inhibit caspase-9 or caspase-7 can be tested using the methods disclosed in Examples section, below.

Cell-Penetrating Caspase-9 or Caspase-7 Inhibitors

In certain embodiments of the present disclosure, the caspase-9 inhibitor or caspase-7 inhibitor is conjugated to a cell penetrating peptide to form a cell-penetrating caspase-9 inhibitor or a cell-penetrating caspase-7 inhibitor.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. In certain embodiments, the cell-penetrating peptide used in the membrane-permeable complex of the present disclosure preferably comprises at least one non-functional cysteine residue, which is either free or derivatized to form a disulfide link with the caspase-9 inhibitor or caspase-7 inhibitor, which has been modified for such linkage. Representative amino acid motifs conferring such properties are listed in U.S. Pat. No. 6,348,185, the contents of which is expressly incorporated herein by reference. Several suitable exemplary cell-penetrating peptides are described by Pescina et al. (2018) Journal of Controlled Release 284:84-102, the disclosure of which is incorporated herein by reference. The cell-penetrating peptides of the present disclosure may include, but are not limited to, Penetratin1, transportan, pIs1, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

The cell-penetrating peptides of the present disclosure include those sequences that retain certain structural and functional features of the identified cell-penetrating peptides, yet differ from the identified peptides' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions, as described above in connection with peptide caspase-9 inhibitors or caspase-7 inhibitors. In certain embodiments, a cell-penetrating peptide of the present disclosure is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the identified peptide and is capable of mediating cell penetration. The effect of the amino acid substitutions on the ability of the synthesized peptide to mediate cell penetration can be tested using the methods disclosed in Examples section, below.

In certain embodiments of the present disclosure, the cell-penetrating peptide is Penetratin1, comprising the peptide sequence C(NPys)-RQIKIWFQNRRMKWKK (SEQ ID NO: 3), or a conservative variant thereof. As used herein, a “conservative variant” is a peptide having one or more amino acid substitutions, wherein the substitutions do not adversely affect the shape—or, therefore, the biological activity (i.e., transport activity) or membrane toxicity—of the cell-penetrating peptide.

Penetratin1 is a 16-amino-acid polypeptide derived from the third alpha-helix of the homeodomain of Drosophila antennapedia. Its structure and function have been well studied and characterized: Derossi et al., Trends Cell Biol., 8(2):84-87, 1998; Dunican et al., Biopolymers, 60(1):45-60, 2001; Hallbrink et al., Biochim. Biophys. Acta, 1515(2):101-09, 2001; Bolton et al., Eur. J. Neurosci., 12(8):2847-55, 2000; Kilk et al., Bioconjug. Chem., 12(6):911-16, 2001; Bellet-Amalric et al., Biochim. Biophys. Acta, 1467(1):131-43, 2000; Fischer et al., J. Pept. Res., 55(2): 163-72, 2000; Thoren et al., FEBS Lett., 482(3):265-68, 2000.

It has been shown that Penetratin1 efficiently carries avidin, a 63-kDa protein, into human Bowes melanoma cells (Kilk et al., Bioconjug. Chem., 12(6):911-16, 2001). Additionally, it has been shown that the transportation of Penetratin1 and its cargo is non-endocytotic and energy-independent, and does not depend upon receptor molecules or transporter molecules. Furthermore, it is known that Penetratin1 is able to cross a pure lipid bilayer (Thoren et al., FEBS Lett., 482(3):265-68, 2000). This feature enables Penetratin1 to transport its cargo, free from the limitation of cell-surface-receptor/-transporter availability. The delivery vector previously has been shown to enter all cell types (Derossi et al., Trends Cell Biol., 8(2):84-87, 1998), and effectively to deliver peptides (Troy et al., Proc. Natl. Acad. Sci. USA, 93:5635-40, 1996) or antisense oligonucleotides (Troy et al., J. Neurosci., 16:253-61, 1996; Troy et al., J. Neurosci., 17:1911-18, 1997) or siRNA (Davidson, T. J. et al., J Neurosci. 2004 Nov. 10; 24(45): 10040-6).

Other non-limiting embodiments of the present disclosure involve the use of the following exemplary cell permeant molecules: RL16 (H-RRLRRLLRRLLRRLRR-OH) (SEQ ID NO: 4), a sequence derived from Penetratin1 with slightly different physical properties (Biochim Biophys Acta. 2008 July-August; 1780(7-8):948-59); and RVG-RRRRRRRRR (SEQ ID NO: 5), a rabies virus sequence which targets neurons see P. Kumar, H. Wu, J. L. McBride, K. E. Jung, M. H. Kim, B. L. Davidson, S. K. Lee, P. Shankar and N. Manjunath, Transvascular delivery of small interfering RNA to the central nervous system, Nature 448 (2007), pp. 39-43.

Transportan is a 27-amino-acid long peptide containing 12 functional amino acids from the amino terminus of the neuropeptide galanin, and the 14-residue sequence of mastoparan in the carboxyl terminus, connected by a lysine (Pooga et al., FASEB J., 12(1):67-77, 1998). It includes the amino acid sequence GWTLNSAGYLLGKINLKALAALAKKIL (SEQ ID NO: 6), or a conservative variant thereof.

pIs1 is derived from the third helix of the homeodomain of the rat insulin 1 gene enhancer protein (Magzoub et al., Biochim. Biophys. Acta, 1512(1):77-89, 2001; Kilk et al., Bioconjug. Chem., 12(6):911-16, 2001). pIs1 includes the amino acid sequence PVIRVW FQNKRCKDKK (SEQ ID NO: 7), or a conservative variant thereof.

Tat is a transcription activating factor, of 86-102 amino acids, that allows translocation across the plasma membrane of an HIV-infected cell, to transactivate the viral genome (Hallbrink et al., Biochem. Biophys. Acta., 1515(2):101-09, 2001; Suzuki et al., J. Biol. Chem., 277(4):2437-43, 2002; Futaki et al., J. Biol. Chem., 276(8):5836-40, 2001). A small Tat fragment, extending from residues 48-60, has been determined to be responsible for nuclear import (Vives et al., J. Biol. Chem., 272(25):16010-017, 1997); it includes the amino acid sequence GRKKRRQRRRPPQ (SEQ ID NO: 8), or a conservative variant thereof.

pVEC is an 18-amino-acid-long peptide derived from the murine sequence of the cell-adhesion molecule, vascular endothelial cadherin, extending from amino acid 615-632 (Elmquist et al., Exp. Cell Res., 269(2):237-44, 2001). pVEC includes the amino acid sequence LLIILRRRIRKQAHAH (SEQ ID NO: 9), or a conservative variant thereof.

MTSs, or membrane translocating sequences, are those portions of certain peptides which are recognized by the acceptor proteins that are responsible for directing nascent translation products into the appropriate cellular organelles for further processing (Lindgren et al., Trends in Pharmacological Sciences, 21(3):99-103, 2000; Brodsky, J. L., Int. Rev. Cyt., 178:277-328, 1998; Zhao et al., J. Immunol. Methods, 254(1-2):137-45, 2001). An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides (Lindgren et al., Trends in Pharmacological Sciences, 21(3):99-103, 2000; Morris et al., Nucleic Acids Res., 25:2730-36, 1997). MPS includes the amino acid sequence GALFLGWLGAAGSTMGAWSQPKKKRKV (SEQ ID NO: 10), or a conservative variant thereof.

Polyarginines (Rₙ), such as those containing n=3-12 consecutive arginines, are cationic synthetic polymers of arginine having a flexible, unstructured or random coil structure able to internalize into cells via direct translocation and endocytosis mechanisms (Pescina et al. (2018) Journal of Controlled Release 284:84-102). Polyarginines and conservative variants thereof are suitable cell-penetrating peptides of the present disclosure.

Diatos peptide vector 1047 (DPV1047, Vectocell®, Diatos, France) refers to a polypeptide having the sequence VKRGLKLRHVRPRVTRMDV (SEQ ID NO:17). DPV1047 is a synthetic cationic polypeptide that allows internalization via an endocytosis mechanism (Pescina et al. (2018) Journal of Controlled Release 284:84-102). DPV1047 or conservative variants thereof are suitable cell-penetrating peptides of the present disclosure.

M918 refers to a polypeptide having the sequence MVTVLFRRLRIRRACGPPRVRV (SEQ ID NO:18). M918 is a cationic, primary amphipathic polypeptide derived from p14ARF protein that allows internalization via an endocytosis mechanism (Pescina et al. (2018) Journal of Controlled Release 284:84-102). M918 or conservative variants thereof are suitable cell-penetrating peptides of the present disclosure.

M1073 refers to a polypeptide having the sequence MVRRFLVTLRIRRACGPPRVRV (SEQ ID NO:19). Like M918, M1073 is a cationic, primary amphipathic polypeptide derived from p14ARF protein that allows internalization via an endocytosis mechanism (Pescina et al. (2018) Journal of Controlled Release 284:84-102). M1073 or conservative variants thereof are suitable cell-penetrating peptides of the present disclosure.

BPrPr (1-28) refers to a polypeptide having the sequence MVKSKIGSWILVLFVAMWSDVGLCKKRP (SEQ ID NO:20). BPrPr (1-28) is a cationic, primary amphipathic polypeptide derived from bovine prion protein that allows internalization via macropynocitosis (Pescina et al. (2018) Journal of Controlled Release 284:84-102). BPrPr (1-28) or conservative variants thereof are suitable cell-penetrating peptides of the present disclosure.

MPG refers to a polypeptide having the sequence GALFLGFLGAAGSTMGAWSQPKKKRKV (SEQ ID NO:21). MPG is formed by fusing the nuclear localization sequences (NLSs) of the simian virus 40 (SV40) large T antigen (KKKRKV (SEQ ID NO:22) to the sequence of the HIV glycoprotein 41 (GALFLGFLGAAGSTMGA (SEQ ID NO:23)). Pep-1 refers to a polypeptide having the sequence KETWWETWWTEWSQPKKKRKV (SEQ ID NO:24). Pep-1 is formed by fusing the nuclear localization sequences (NLSs) of the simian virus 40 (SV40) large T antigen (KKKRKV (SEQ ID NO:22) to a tryptophan-rich cluster (KETWWETWWTEW (SEQ ID NO:25)). MPG and Pep-1 are both cationic, primary amphipathic polypeptides that allow internalization via endocytosis-independent mechanisms (Pescina et al. (2018) Journal of Controlled Release 284:84-102). MPG, Pep-1 or conservative variants thereof are suitable cell-penetrating peptides of the present disclosure.

Model amphipathic peptides, or MAPs, form a group of peptides that have, as their essential features, helical amphipathicity and a length of at least four complete helical turns (Scheller et al., J. Peptide Science, 5(4):185-94, 1999; Hallbrink et al., Biochim. Biophys. Acta., 1515(2):101-09, 2001). An exemplary MAP comprises the amino acid sequence KLALKLALKALKAALKLA (SEQ ID NO: 11)-amide, or a conservative variant thereof. Other exemplary MAPs comprise MAP12, having the sequence LKTLTETLKELTKTLTEL (SEQ ID NO:26) which is a synthetic anionic, secondary amphipathic, α-helical peptide that allows internalization via an endocytosis-independent mechanism, and MAP17, having the sequence QLALQLALQALQAALQLA (SEQ ID NO:27), which is an amphipathic, secondary amphipathic, α-helical peptide (Pescina et al. (2018) Journal of Controlled Release 284:84-102). MAP12, MAP17 or conservative variants thereof are suitable cell-penetrating peptides of the present disclosure.

GALA refers to a polypeptide having the sequence WEAALAEALAEALAEHLAEALAEALEALAA (SEQ ID NO:28). GALA is a synthetic, anionic, secondary amphipathic polypeptide (Pescina et al. (2018) Journal of Controlled Release 284:84-102). GALA or conservative variants thereof are suitable cell-penetrating peptides of the present disclosure.

p28 refers to a polypeptide having the sequence LSTAADMQGVVTDGMASGLDKDYLKPDD (SEQ ID NO:29). p28 is an anionic, secondary amphipathic peptide derived from the bacterial protein azurin. p28 allows internalization via a caveolae-mediated mechanism (Pescina et al. (2018) Journal of Controlled Release 284:84-102). p28 or conservative variants thereof are suitable cell-penetrating peptides of the present disclosure.

PreS2 refers to an amphipathic peptide having the sequence PLSSIFSRIGDP (SEQ ID NO:30) derived from the PreS2-domain of hepatitis-B virus surface antigens (Pescina et al. (2018) Journal of Controlled Release 284:84-102). PreS2 or conservative variants thereof are suitable cell-penetrating peptides of the present disclosure.

VT5 refers to a synthetic, secondary amphipathic peptide having the sequence DPKGDPKGVTVTVTVTVTGKGDPKPD (SEQ ID NO:31) and comprising β-sheet structure (Pescina et al. (2018) Journal of Controlled Release 284:84-102). VT5 or conservative variants thereof are suitable cell-penetrating peptides of the present disclosure.

Bac (1-24) refers to a polypeptide having the sequence RRIRPRPPRLPRPRPRPLPFPRPG (SEQ ID NO:32), comprising residues 1-24 of bactenecin-7 (Bac7), a 59-residue antimicrobial protein. Bac (1-24) is a cationic, polyproline II helical polypeptide that allows internalization via endocytosis mechanisms (Pescina et al. (2018) Journal of Controlled Release 284:84-102). Bac (1-24) or conservative variants thereof are suitable cell-penetrating peptides of the present disclosure.

PPR and PRR refer to synthetic, cationic, proline-rich polypeptides having the sequence (PPR)ₙ and (PRR)ₙ, respectively, wherein n=3-6, and having a polyproline II helical structure (Pescina et al. (2018) Journal of Controlled Release 284:84-102). PPR, PRR or conservative variants thereof are suitable cell-penetrating peptides of the present disclosure.

SAP refers to an amphipathic, polyproline II helical polypeptide based on a γ-zein sequence. SAP has the sequence VRLPPPVRLPPPVRLPPP (SEQ ID NO:33). SAP(E) refers to a synthetic, anionic, polyproline II helical polypeptide variant of SAP having the sequence VELPPPVELPPPVELPPP (SEQ ID NO:34). SAP and SAP(E) allow internalization via an endocytosis mechanism (Pescina et al. (2018) Journal of Controlled Release 284:84-102). SAP, SAP(E) or conservative variants thereof are suitable cell-penetrating peptides of the present disclosure.

CyLoP-1 refers to an amphipathic polypeptide having the sequence CRWRWKCCKK (SEQ ID NO:35) derived from the nuclear localization domain crot (27-39) of the snake venom toxin crotamine (Pescina et al. (2018) Journal of Controlled Release 284:84-102). CyLoP-1 or conservative variants thereof are suitable cell-penetrating peptides of the present disclosure.

gH 625 refers to a hydrophobic, α-helical polypeptide having the sequence HGLASTLTRWAHYNALIRAF (SEQ ID NO:36), derived from Herpes simplex virus type I (Pescina et al. (2018) Journal of Controlled Release 284:84-102). gH 625 or conservative variants thereof are suitable cell-penetrating peptides of the present disclosure.

CPP-C refers to a hydrophobic polypeptide having the sequence PIEVCMYREP (SEQ ID NO:37) derived from the C-terminal region (residues 140-149) of the fibroblast-growth factor 12 (FGF12) (Pescina et al. (2018) Journal of Controlled Release 284:84-102). CPP-C or conservative variants thereof are suitable cell-penetrating peptides of the present disclosure.

C105Y refers to a synthetic hydrophobic polypeptide having the sequence CSIPPEVKFNKPFVYLI (SEQ ID NO:38) derived from amino acid sequences corresponding to residues 359-374 and a C-terminal domain of alpha-1-antitrypsin (Pescina et al. (2018) Journal of Controlled Release 284:84-102). C105Y or conservative variants thereof are suitable cell-penetrating peptides of the present disclosure.

Pep-7 and SG3 refer to hydrophobic polypeptides obtained from randomized peptide libraries using phage display- or plasmid display-based functional selection platforms, respectively (Pescina et al. (2018) Journal of Controlled Release 284:84-102). Pep-7 has the sequence SDLWEMMMVSLACQY (SEQ ID NO:39) and SG3 has the sequence RLSGMNEVLSFRW (SEQ ID NO:40). Pep-7, SG3 or conservative variants thereof are suitable cell-penetrating peptides of the present disclosure.

In certain embodiments, the cell-penetrating peptides and the caspase-9 inhibitors or caspase-7 inhibitors described above are covalently bound. In certain embodiments the cell-penetrating peptide is operably linked to a peptide caspase-9 inhibitor or caspase-7 inhibitor via recombinant DNA technology. For example, in embodiments where the caspase-9 or caspase-7 inhibitor is a peptide caspase-9 or caspase-7 inhibitor, a nucleic acid sequence encoding that peptide caspase-9 or caspase-7 inhibitor can be introduced either upstream (for linkage to the amino terminus of the cell-penetrating peptide) or downstream (for linkage to the carboxy terminus of the cell-penetrating peptide), or both, of a nucleic acid sequence encoding the cell-penetrating peptide of interest. Such fusion sequences including both the peptide caspase-9 inhibitor-encoding nucleic acid sequence and the cell-penetrating peptide-encoding nucleic acid sequence can be expressed using techniques well known in the art.

In certain embodiments the caspase-9 or caspase-7 inhibitor can be operably linked to the cell-penetrating peptide via a non-covalent linkage. In certain embodiments such non-covalent linkage is mediated by ionic interactions, hydrophobic interactions, hydrogen bonds, or van der Waals forces.

In certain embodiments the caspase-9 or caspase-7 inhibitor is operably linked to the cell penetrating peptide via a chemical linker. Examples of such linkages typically incorporate 1-30 nonhydrogen atoms selected from the group consisting of C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In certain embodiments, the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. In certain embodiments, the linker is a combination of single carbon-carbon bonds and carboxamide, sulfonamide or thioether bonds.

A general strategy for conjugation involves preparing the cell-penetrating peptide and the caspase-9 or caspase-7 inhibitor components separately, wherein each is modified or derivatized with appropriate reactive groups to allow for linkage between the two. The modified caspase-9 or caspase-7 inhibitor is then incubated together with a cell-penetrating peptide that is prepared for linkage, for a sufficient time (and under such appropriate conditions of temperature, pH, molar ratio, etc.) as to generate a covalent bond between the cell-penetrating peptide and the caspase-9 or caspase-7 inhibitor.

Numerous methods and strategies of conjugation will be readily apparent to one of ordinary skill in the art, as will the conditions required for efficient conjugation. By way of example only, one such strategy for conjugation is described below, although other techniques, such as the production of fusion proteins or the use of chemical linkers is within the scope of the present disclosure.

In certain embodiments, when generating a disulfide bond between the caspase-9 or caspase-7 inhibitor and the cell-penetrating peptide of the present disclosure, the caspase-9 or caspase-7 inhibitor can be modified to contain a thiol group, and a nitropyridyl leaving group can be manufactured on a cysteine residue of the cell-penetrating peptide. Any suitable bond (e.g., thioester bonds, thioether bonds, carbamate bonds, etc.) can be created according to methods generally and well known in the art. Both the derivatized or modified cell-penetrating peptide, and the modified caspase-9 or caspase-7 inhibitor are reconstituted in RNase/DNase sterile water, and then added to each other in amounts appropriate for conjugation (e.g., equimolar amounts). The conjugation mixture is then incubated for 60 min at 37° C., and then stored at 4° C. Linkage can be checked by running the vector-linked caspase-9 inhibitor molecule, and an aliquot that has been reduced with DTT, on a 15% non-denaturing PAGE. Caspase-9 or caspase-7 inhibitor molecules can then be visualized with the appropriate stain.

In certain embodiments, the present disclosure is directed to a Penetratin1-XBIR3 (Pen1-XBIR3) conjugate in which the caspase-9 inhibitor and the cell-penetrating peptide are linked by a disulfide bond. In certain of such embodiments, the sequence of the Pen-1-XBIR3 is: C(NPys)-RQIKIWFQNRRMKWKK-s-s-MGSSHHHHHSSGLVPRGSHMSTNTCLPRNPSMADYEARIFTFGTWIYSVNKEQ LARAGFYTDWALGEGDKVKCFHCGGGLRPSEDPWEQHARWYPGCRYLLEQRG QEYINNIHLTHS (SEQ ID NO:12). In other of such embodiments, the sequence of the Pen1-XBIR3 is: C(NPys)-RQIKIWFQNRRMKWKK-s-s-MGSSHHHHHHSSGLVPRGSHMSTNTLPRNPSMADYEARIFTFGTWIYSVNKEQL ARAGFYTDWALGEGDKVKCFHCGGGLRPSEDPWEQHARWYPGCRYLLEQRGQ EYINNIHLTHS (SEQ ID NO:13). In other of such embodiments, the His-tag comprised in SEQ ID NO:12 and SEQ ID NO:13 can be removed such that the respective Pen-1-XBIR3 lacks a His-tag.

In some embodiments, the sequence of the Pen1-XBIR3 is:

(SEQ ID NO: 41)
C(NPys)-RQIKIWFQNRRIVIKWKK-s-s-
MSTNLPRNPSMADYEARIFTFGTWIYSVNKEQ
LARAGFYALGEGDKVKCFHCGGGLTDWKPSEDPWEQHAKWYPGCKYLLE
QKGQEYINNIHLTHS.

In certain embodiments, the present disclosure is directed to a Penetratin1 1-XBIR2 (Pen1-XBIR2) conjugate in which the caspase-7 inhibitor and the cell-penetrating peptide are linked by a disulfide bond. In certain of such embodiments, the sequence of the Pen-1-XBIR2 is:

(SEQ ID NO: 42)
C(NPys)-RQIKIWFQNRRIVIKWKK-s-s-
MEEARLKSFQNWPDYAHLTPRELASAGLYY
TGIGDQVQCFCCGGKLKNWEPCDRAWSEHRRHFPNCFFV.

Pharmaceutical Compositions

To facilitate delivery to a cell, tissue, or patient, the cell-penetrating caspase-9 or caspase-7 inhibitor of the present disclosure may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the cell-penetrating caspase-9 or caspase-7 inhibitor or the biological activity of the recipient of the composition. Suitable pharmaceutical carriers, excipients, and/or diluents for use in the present disclosure include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water. Specific formulations of compounds for therapeutic treatment are discussed in Hoover, J. E., Remington's Pharmaceutical Sciences (Easton, Pa.: Mack Publishing Co., 1975) and Liberman and Lachman, eds. Pharmaceutical Dosage Forms (New York, N.Y.: Marcel Decker Publishers, 1980).

In accordance with the methods of the present disclosure, the quantity of the cell-penetrating caspase-9 or caspase-7 inhibitor that is administered to a cell, tissue, or patient should be an effective amount.

### EXAMPLES

The following examples are provided for illustrative purposes only. They are not intended to and should not be interpreted as disclosing the entire breadth of the invention. Furthermore, although the Examples include specific details, their teaching are applicable to and combinable with other details of the disclosure from other examples or other portions of this specification unless such combinations are clearly mutually exclusive.

### Example 1: Retinal Models for Neurodegeneration

These examples relate to testing and results obtained by evaluating the retina of the eye, but are applicable to other tissues, in particular central nervous system tissues, such as the brain or spinal column because of similarities between brain, spinal, and retina neurons, as well as significant physiological similarities, in particular the presence of a barrier, commonly referred to as the “blood-retina barrier” between retinal blood vessels and retinal neurons, which is highly similar to the blood-brain barrier and the blood-spinal column barrier.

Live imaging and electrophysiology can be used to follow retinal function non-invasively in people and rodents. This renders the retina neurons an accessible in vivo model for studying neurodegeneration, as has been recognized in various scientific publications, such as A. London, I. Benhar, M. Schwartz, The retina as a window to the brain-from eye research to CNS disorders. Nature reviews. Neurology 9, 44-53 (2013).

The present examples use a mouse model of retinal vein occlusion (RVO), which results in retinal hypoxia. Mouse retina vein occlusion model are also recognized and describe in various scientific publications, such as A. Ebneter, C. Agca, C. Dysli, M. S. Zinkernagel, Investigation of retinal morphology alterations using spectral domain optical coherence tomography in a mouse model of retinal branch and central retinal vein occlusion. PloS one 10, e0119046 (2015); S. Fuma et al., A pharmacological approach in newly established retinal vein occlusion model. Scientific reports 7, 43509 (2017); H. Zhang et al., Development of a new mouse model of branch retinal vein occlusion and retinal neovascularization. Japanese journal of ophthalmology 51, 251-257 (2007); and Martin, G., Conrad, D., Cakir, B., Schlunck, G., and Agostini, H. T. (2018). Gene expression profiling in a mouse model of retinal vein occlusion induced by laser treatment reveals a predominant inflammatory and tissue damage response. PloS one 13, e0191338.

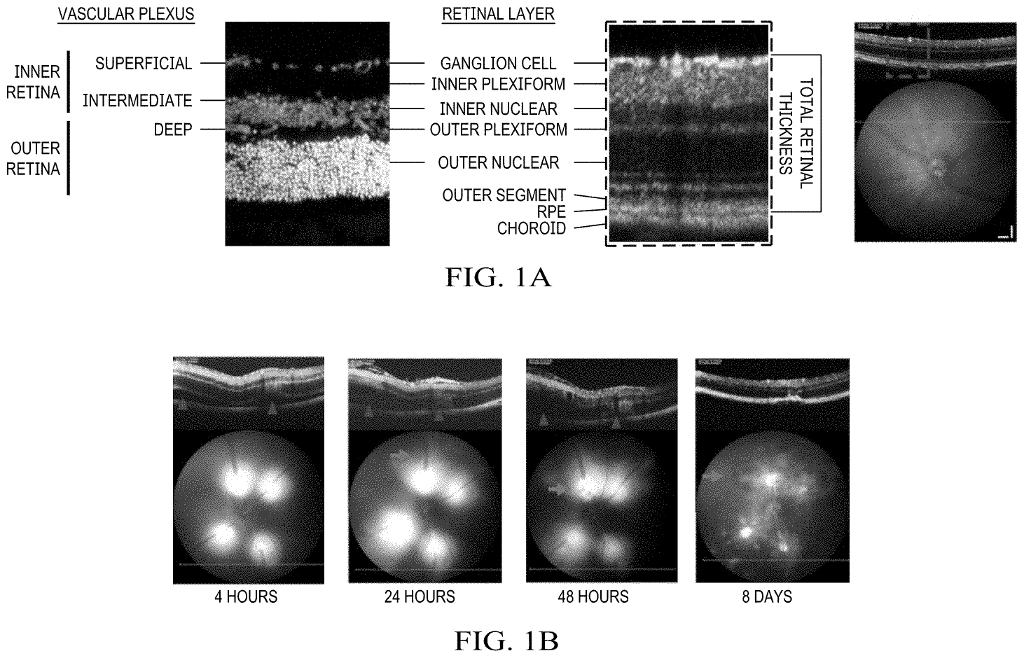

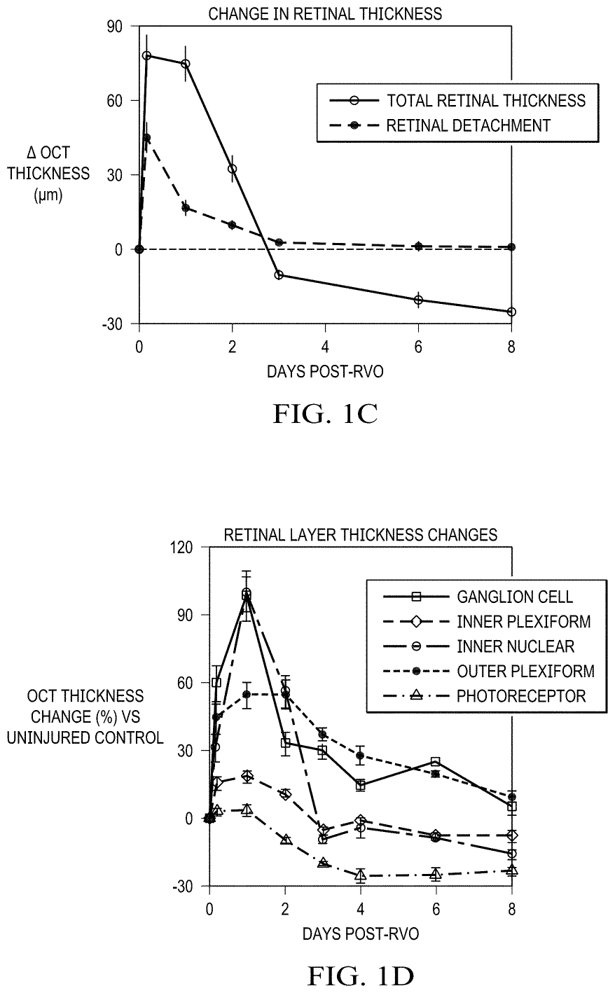

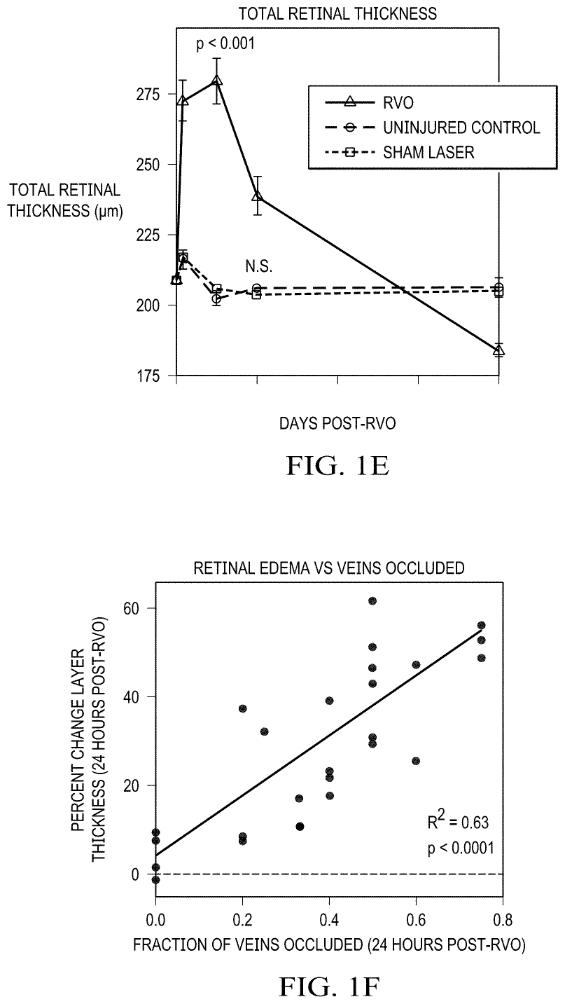

Live retinal imaging in this mouse model allowed measurement of retinal vessels in the vascular plexus (FIG. 1A, left panel)) and of retinal features in the retinal layers (FIG. 1A, middle panel; right panel). This measurement capability facilitated close tracking of the temporal progression of edema and cell loss through distinct retinal layers. Previous studies have measured correlation between visual acuity and OCT measures of retinal thickness in diabetic macular edema and RVO patients (Grewal et al. (2017) American journal of ophthalmology 177, 116-125; Kang et al. (2014) Albrecht von Graefes Archiv fur klinische and experimentelle Ophthalmologie 252, 1413-1421; Ou et al. (2017) American journal of ophthalmology 180, 8-17). While total retinal thickness is the most commonly used clinical measure, layer-specific changes, including outer retinal thickness, inner retinal thickness, and retinal detachment, have shown independent correlation with visual acuity in patients with retinal vascular disease (Grewal et al. (2017) American journal of ophthalmology 177, 116-125; Vujosevic et al. (2017) Acta ophthalmologica 95, 464-471; Wong et al. (2015) BioMed research international 2015, 981471.

Following induction of RVO by laser photocoagulation, vessel dilation and hemorrhaging were observed (FIG. 1B), consistent with ophthalmological findings in RVO patients (Ho et al. Retina (Philadelphia, Pa.) 36, 432-448; Jaulim et al. (2013) Retina (Philadelphia, Pa.) 33, 901-910). Longitudinal OCT imaging of the same eye over 8 days shows the typical progression of retinal pathology caused by RVO; accumulation of subretinal fluid leads to extensive retinal detachment within hours of occlusion, while intraretinal edema peaks by 24-48 hrs post-RVO (FIG. 1B and 1C). By 48 hrs post-RVO, there is clear disorganization of inner retinal layers, and retinal atrophy is apparent 8 days post-RVO. Composite OCT thickness analyses from 13-21 eyes per time-point show rapid induction of retinal swelling and detachment, which resolve by 72 hrs post-RVO (FIG. 1C).

Retinal vasculature supplies blood flow to all layers of the inner retina—the ganglion cell layer (GCL), the inner plexiform layer (IPL) and the inner nuclear layer (INL)—and to the outer plexiform layer (OPL) (FIG. 1A). Analysis of RVO-induced changes revealed that edema is driven primarily by swelling of the GCL, INL, and OPL (FIG. 1D). In contrast, the photoreceptor/outer nuclear layer (ONL) is avascular and is not subject to vasogenic edema; minimal swelling from cytotoxic edema occurs 4-24 hrs after RVO. Retinal detachment peaks during the acute edema phase of RVO as fluid accumulates in the subretinal space, and resolves by 8 days post-RVO. Retinal detachment uncouples photoreceptors from choroidal circulation, resulting in ischemia and photoreceptor degeneration (Yang et al. (2004). Investigative ophthalmology & visual science 45, 648-654. Retinal ischemic injury induces cell death in retinal neurons in the inner and outer nuclear layers (Nishijima et al. (2007) The American journal of pathology 171, 53-67. Retinal atrophy was observed post-RVO, driven by thinning of the INL and photoreceptor layers (FIG. 1D).

### Example 2: Intraretinal Swelling Correlates with Degree of Retinal Vein Occlusion

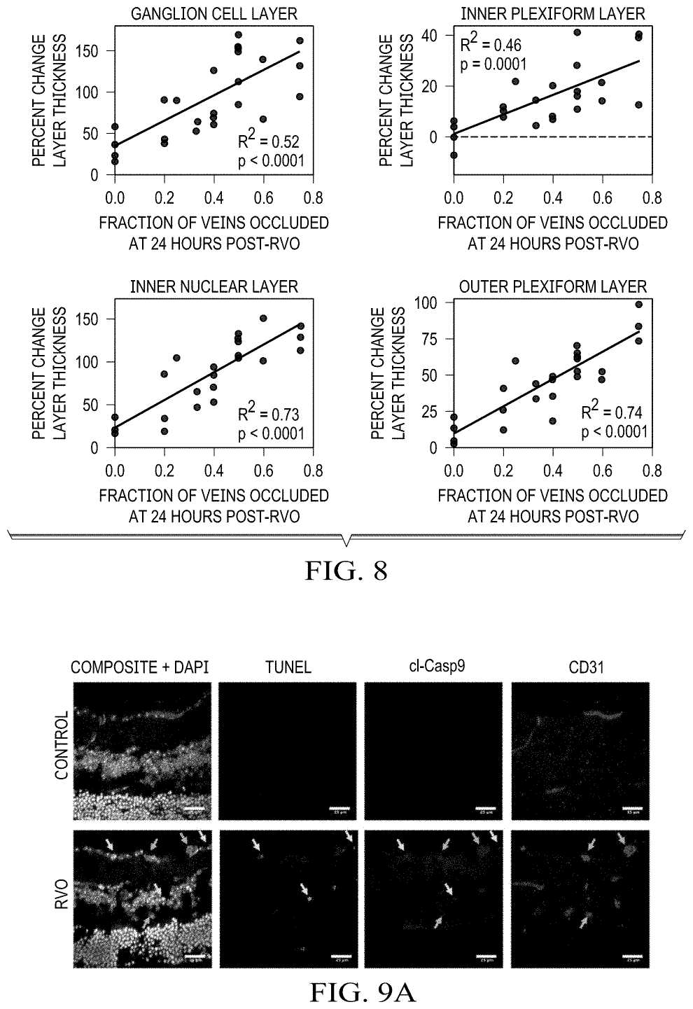

A potential confounding feature of the laser-induced RVO model is the direct retinal injury from the laser burns, which trigger a tissue damage response (Martin et al. (2018) PloS one 13, e0191338). To minimize this influence on the analyses, the laser intensity and number of laser applications per vessel were limited, and retinal injury was measured distal from the burn sites. To further discriminate between retinal layer changes caused by laser injury versus ischemic insult, retinal thickness was compared between laser-induced RVO, uninjured control eyes, and eyes treated with sham laser condition where laser burns were applied to the capillary bed between major retinal veins (FIG. 1E). Total intraretinal swelling, and swelling of the GCL, IPL, INL, and OPL were highly correlated with the fraction of veins which remained occluded at 24 hrs post-RVO (FIG. 1F, FIG. 8).

### Example 3: RVO Induces Neuronal, But Not Endothelial Cell Death

Tight junctions between endothelial cells help form the blood-retina barrier, which controls fluid and molecular movement within retinal tissues. RVO triggers inflammatory and hypoxia responses in the retina, both of which contribute to breakdown of the blood-retina barrier. To determine if endothelial cell death played a role in blood-retina barrier breakdown after RVO, retinal flatmounts were immunostained for Ph2AX (a marker of dying cells), cleaved caspase-9 (an initiator of apoptosis), and CD31 (an endothelial marker) at different time points after RVO.

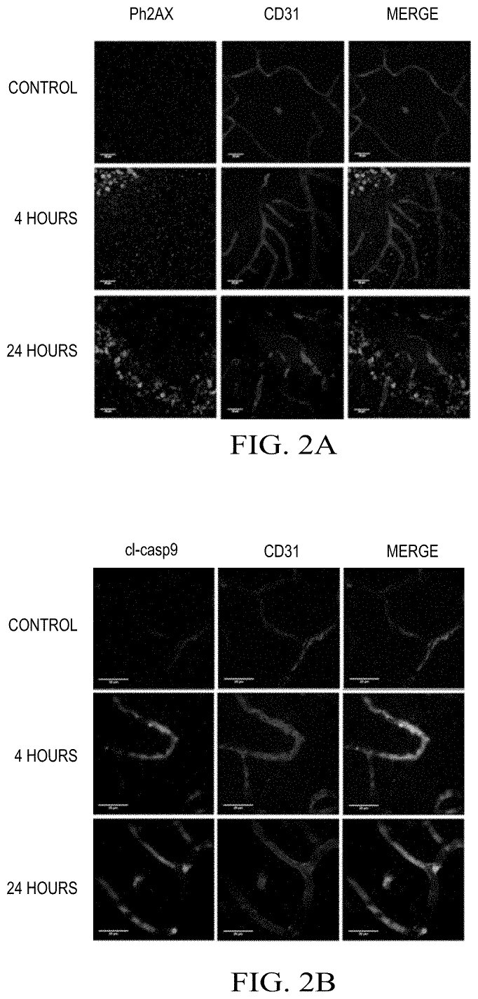

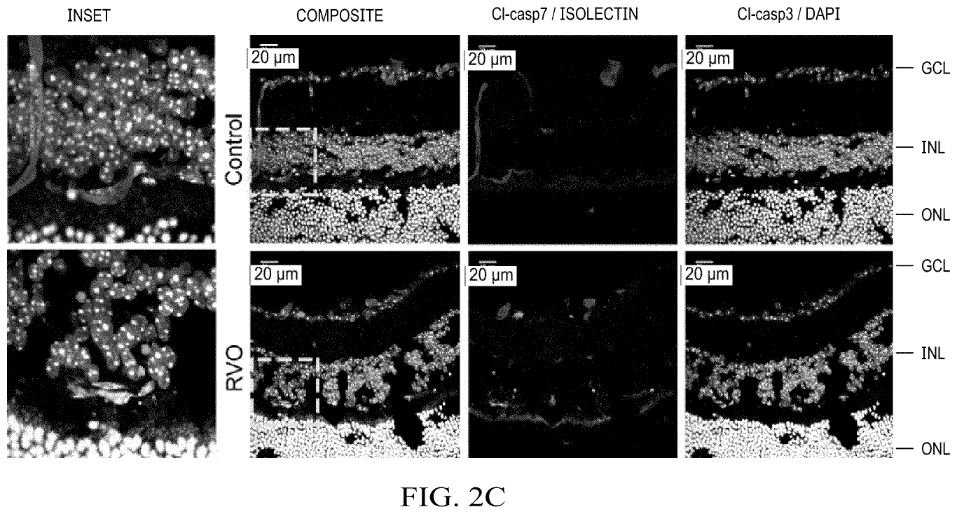

Ph2AX was induced by 4 hours post-RVO, but did not co-localize with CD31 through 24 hrs (FIG. 2A).

Surprisingly, by 4 hours post-RVO cleaved caspase-9 co-localized with CD31 in the capillary bed distal to the site of RVO, suggesting that RVO induced endothelial caspase-9 activation but not endothelial cell death (FIG. 2B). Cleaved caspase-9 continued to be evident in vessels at 24 hours.

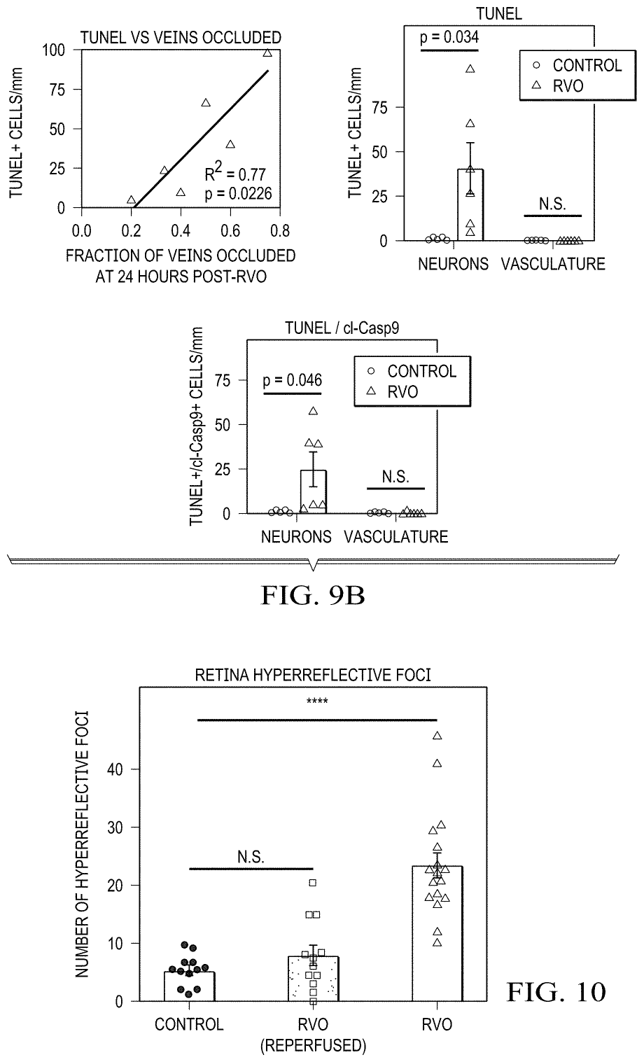

To determine if RVO-induced activation of caspase-9 was associated with cell death, TUNEL staining was utilized to mark the nuclei of dying cells, and the colocalization of TUNEL and cl-Casp9 was evaluated in neuronal cell layers and in endothelial cells (FIG. 9A, FIG. 9B). The induction of TUNEL correlates with the fraction of veins occluded at 24 hr post-RVO. RVO induces a significant increase in TUNEL, as well as in colocalization of cl-Casp9 and TUNEL in neuronal cells, but not in the vasculature, indicating that RVO induces cell-type specific regulation of caspase-9 activity, and that caspase-9 is not causing death in endothelial cells.

While hypoxic stimulus may induce cleavage of caspase-9, endogenous inhibitors of apoptosis proteins modulate its activity within the cell (Denault, et al. (2007) The Biochemical journal 405, 11-19; Garnier et al. (2004) The European journal of neuroscience 20, 937-946. To test if cleaved caspase-9 was active, retinal cross sections were immunostained for cleavage of its target substrates, caspase-3 and caspase-7 (FIG. 2C). Induction of cleaved caspase-7 was detected in both endothelial and neuronal cells, but not cleaved caspase-3, which remained at low basal levels. Based on the activation of endothelial caspase-7 and the absence of notable endothelial cell death, it was hypothesized that a non-death role for caspase-9 in endothelial cell dysfunction may be a therapeutic target for RVO.

### Example 4: Eye Drops Deliver a Specific Inhibitor of Caspase-9, Pen1-XBir3, to the Retina

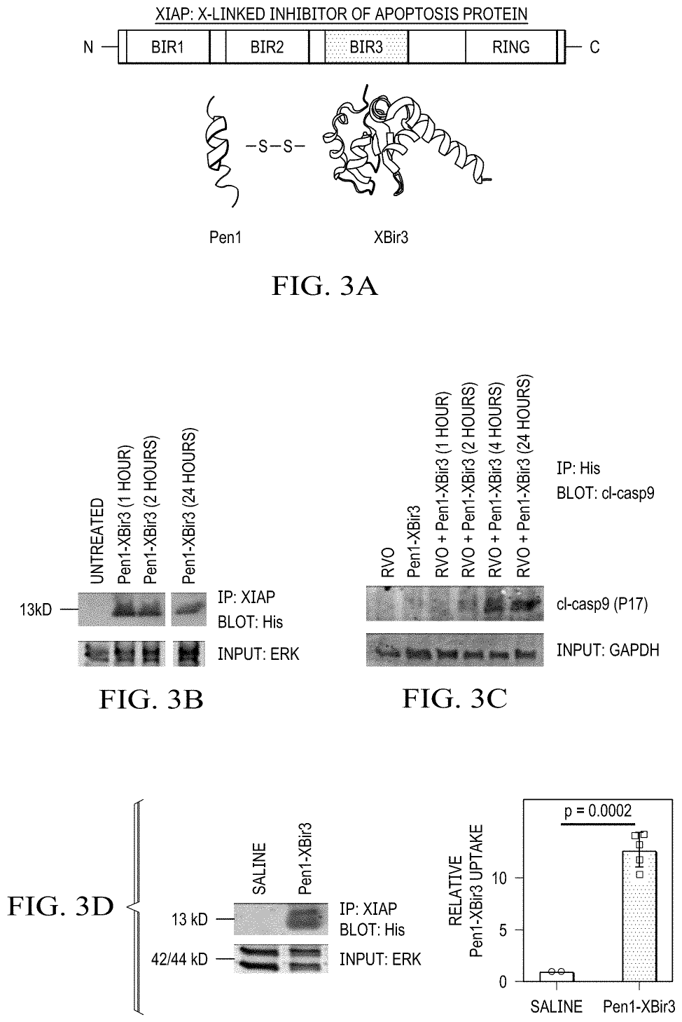

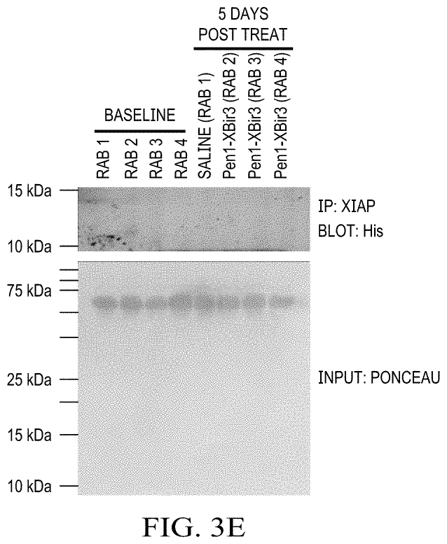

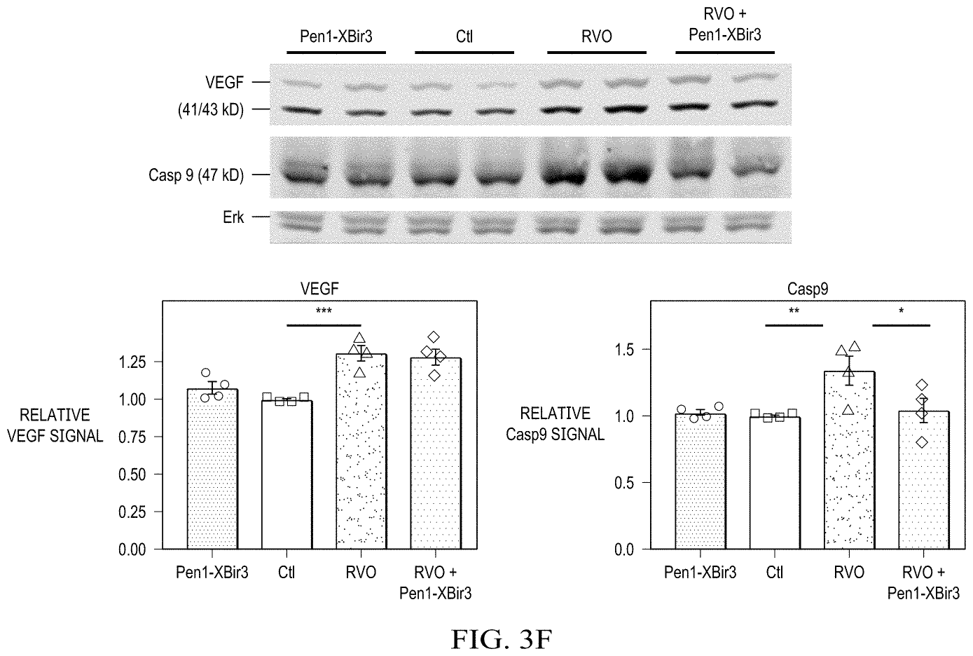

To interrogate the functional significance of caspase-9 activity in RVO, tests were conducted with Pen1-XBir3, a cell penetrating specific inhibitor of caspase-9. Pen1 was cross-linked to XBir3 with a disulfide bond which is reduced in the cytoplasm (FIG. 3A), allowing XBir3 to effectively inhibit caspase-9 unimpeded by Pen1.

To determine if topical application of Pen1-XBir3 would deliver XBir3 to the retina, mice were treated with eye drops containing 10 μg Pen1-XBir3, and retinas were harvested for analysis at the indicated time points (FIG. 3B). Retinal lysates were immunoprecipitated with an XIAP antibody generated to the BIR3 domain followed by western blot for His (the XBir3 used in this Example contains a His-tag). XBir3 was present in retinal lysates at 1 hour post-treatment and was still detectable in the retina at 24 hours post-treatment.

### Example 5: Immunoprecipitation Detection of Intraretinal Target Engagement by Pen1-XBir3 Eye Drops

XBir3 targets auto-cleaved caspase-9. To determine whether XBir3 binds caspase-9 in the retina, mice were subjected to RVO, with or without treatment with Pen1-XBir3 eye drops immediately after RVO. Retinas were harvested at the indicated times for analysis. Immunoprecipitation of His followed by western blot for caspase-9 showed that XBir3 and its target caspase-9 co-precipitated by 2 hrs post-RVO and target engagement was still evident at 24 hrs post-RVO, confirming target engagement (FIG. 3C).

### Example 6: Topical Delivery of Pen1-XBir3 in Rabbit Eyes

To test if topical delivery of Pen1-XBir3 is potentially scalable to clinical applications, retinal uptake of Pen1-XBir3 eye drops in rabbits was tested; rabbit and human eyes have similar pharmacokinetic parameters, making the rabbit eye a good model for ocular pharmacokinetics. Pen1-XBir3 eye-drops were administered twice-daily to rabbits for 5 consecutive days. XBir3 was detected in retinal lysates, but not in the blood plasma of treated rabbits (FIG. 3D and 3E), supporting local delivery of XBir3 to the retina.

### Example 7: Pen1-XBir3 blocks increase in caspase-9 expression but does not change VEGF Expression

In addition to inducing cleavage of caspase-9, hypoxia and ischemia increase expression of total caspase-9 in central nervous system tissues. Total levels of caspase-9 were increased 24 hours after RVO and this increase was blocked by Pen1-XBir3 eye-drops (FIG. 3F). Hypoxia also induces the expression of VEGF, which is the main target of current therapies for RVO. There was an increase in VEGF levels 24 hours after RVO, but this increase was not affected by Pen1-XBir3 eye drops (FIG. 3F), suggesting that a different pathway of vascular dysfunction was targeted by the caspase-9 inhibitor.

### Example 8: Pen1-XBir3 Eye-Drops Block Activation of Caspase-9 and Caspase-7

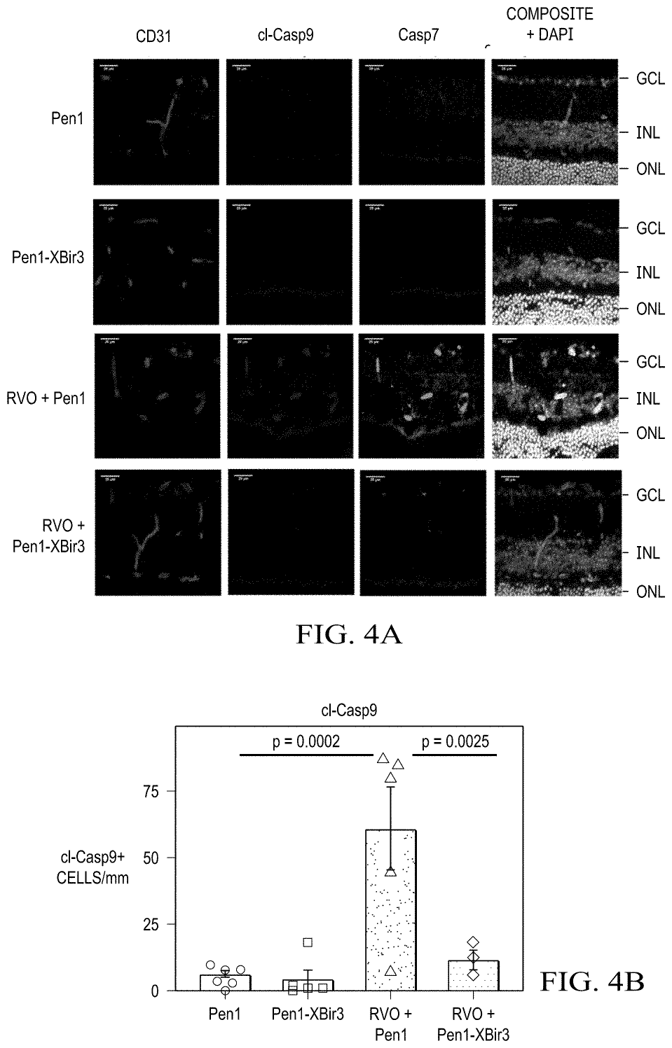

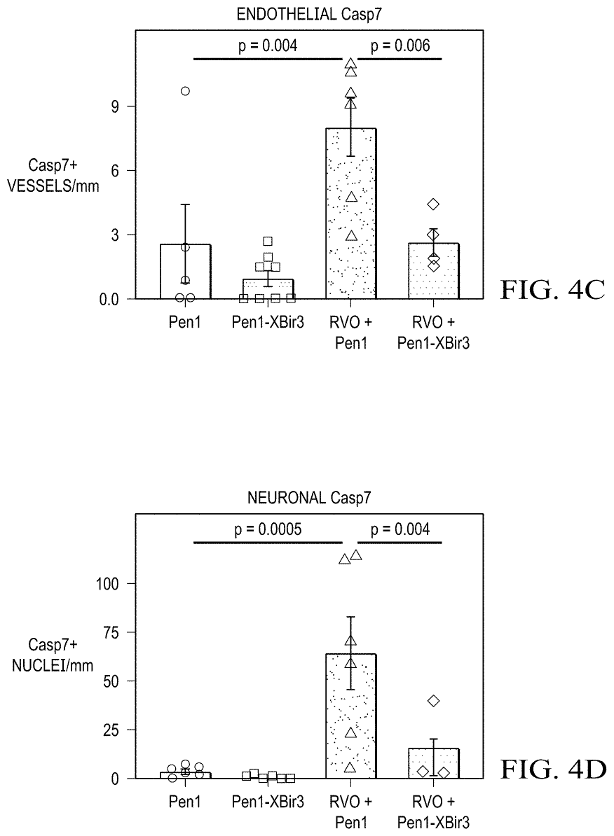

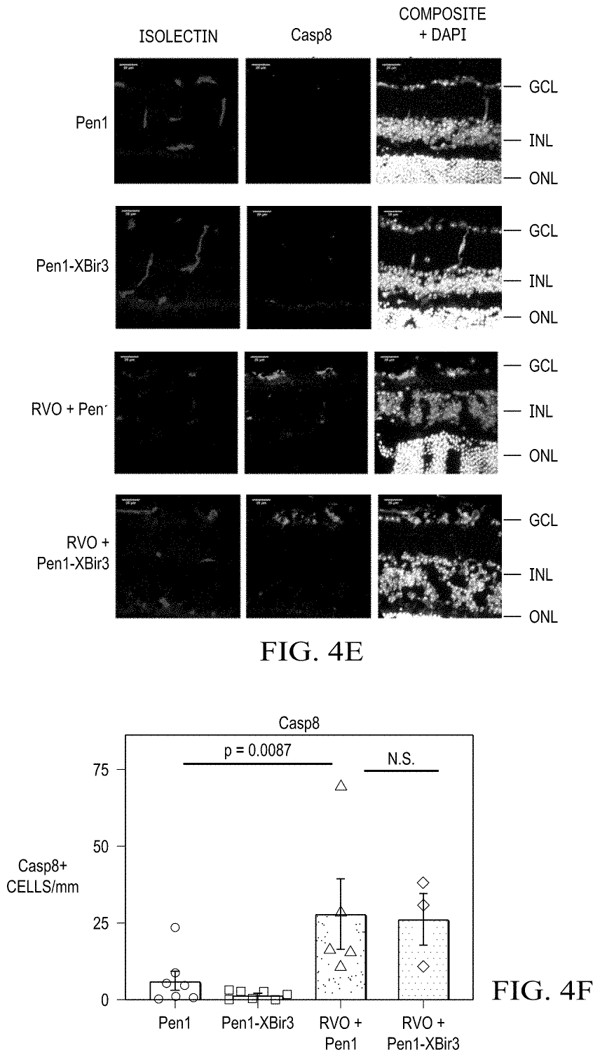

To measure the effect of Pen1-XBir3 on caspase-9 activity in retinal tissues, retinal sections were immunostained for cleaved caspase-9, caspase-7, and CD31 (FIG. 4A). Cleaved caspase-9 and caspase-7 co-localized in endothelial cells and in retinal neurons after RVO. Treatment with Pen1-XBir3 eye-drops immediately after RVO blocked the autocleavage of caspase-9 and the induction of caspase-7 in both endothelial cells and neurons (FIG. 4A to FIG. 4D), supporting that caspase-9 is the main protease responsible for activation of caspase-7 in RVO. However, residual caspase-7 was detected in some cells in the GCL. To determine if another initiator caspase may be active in ganglion neurons, retinas were immunostained for caspase-8 (FIG. 4E). Caspase-8 was induced in the GCL layer; treatment with Pen1-XBir3 did not modulate expression of caspase-8 (FIG. 4F), indicating that caspase-8 induction is independent of caspase-9 inhibition.

### Example 9: Blocking Caspase-9 Activity Rescues Vascular Integrity after RVO

To investigate the therapeutic potential of caspase-9 inhibition in RVO, live retinal imaging was used to follow the progression of post-RVO retinal pathology in mice which received either Pen1-XBir3 or Pen1 control eye drops. Mice received two doses of eye drops (immediately post-RVO, and at 24 hrs post-RVO) to maintain caspase-9 inhibition throughout the edema phase of RVO injury. Eye drops containing Pen1-saline were used as a vehicle control.

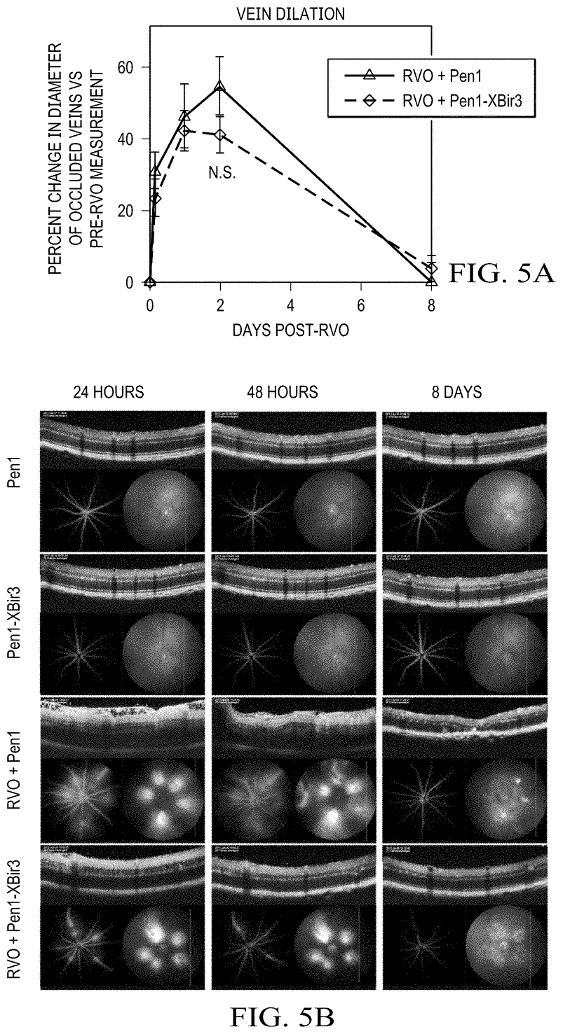

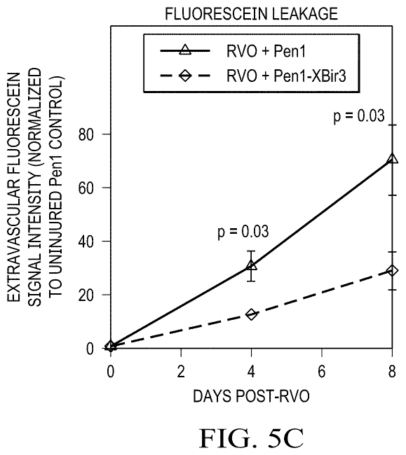

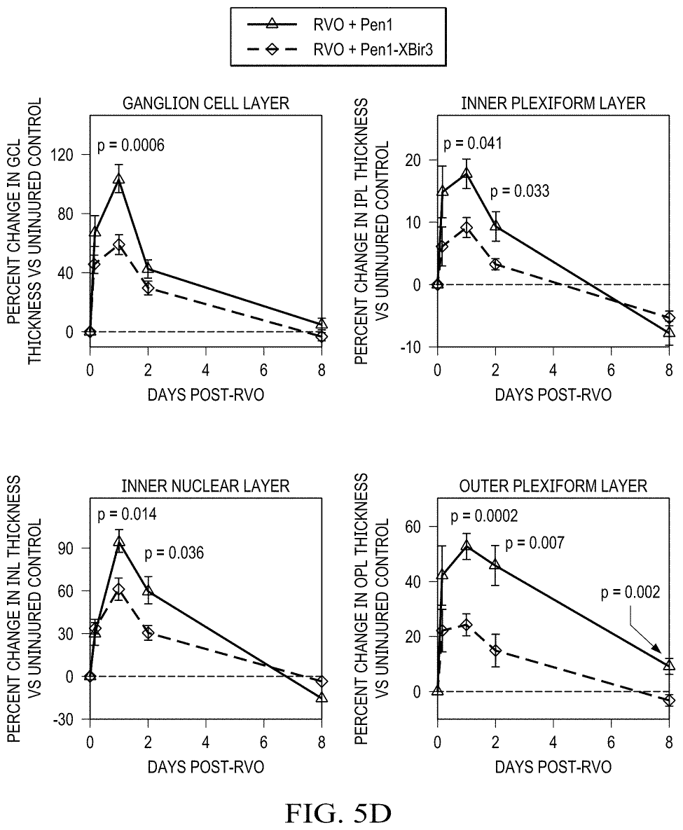

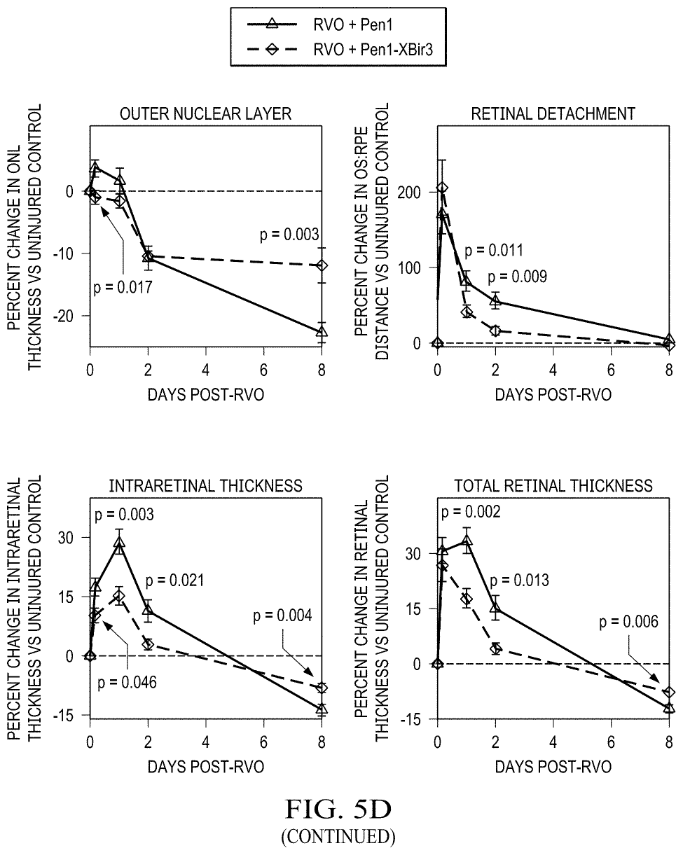

RVO causes an immediate dilation of retinal veins as blood accumulates distal to the site of occlusion (Ebneter et al. (2015) PloS one 10, e0119046). An increase in VEGF levels in response to retinal hypoxia promotes further vasodilation by increasing synthesis of nitric oxide (Ferrara et al. (2003) Nature medicine 9, 669-676; Hood et al. (1998) The American journal of physiology 274, H1054-1058). Vein diameter increased immediately after laser photocoagulation, and reached a maximum at 24-48 hrs, before returning towards baseline as edema resolved (FIG. 5A). Consistent with results showing no impact of Pen1-XBir3 on retinal VEGF levels, treatment with Pen1-XBir3 did not alter the course of vein dilation following RVO.

Fluorescein angiography was used to assess the disruption of retinal barrier function after RVO. Fluorescein is a small fluorescent dye used extensively as a diagnostic tool in ophthalmology. Fluorescein readily enters vascular circulation in peripheral tissues and can be detected in retinal vasculature in a time-dependent manner. In healthy subjects, the BRB confines fluorescein signal to the retinal vasculature; in damaged eyes, fluorescein accumulates in areas where endothelial barrier integrity has been disrupted, indicating vasogenic edema. Increased fluorescein leakage was present 24-48 hrs post-RVO, coinciding with peak retinal swelling as measured by OCT (FIG. 5B, FIG. 5C). Treatment with Pen1-XBir3 substantially reduced the amount of fluorescein leakage in injured eyes, indicating that inhibition of caspase-9 helped preserve blood-retina barrier integrity.

### Example 10. OCT Imaging Measures Protection of Retinal Morphology

Mice treated with Pen1-XBir3 immediately after RVO had significantly less retinal swelling compared to vehicle-treated eyes (FIG. 5B, FIG. 5D). During peak edema at 24 hrs post-RVO, treated mice had 50% less total retinal swelling, 56% less retinal detachment, and 64% less swelling in the OPL. Furthermore, Pen1-XBir3 reduced INL thinning by 77%, and reduced ONL thinning by 48%. Total retinal atrophy was significantly reduced in treated eyes compared to vehicle controls (FIG. 5D).

### Example 11. Caspase-9 Inhibitor Reduces Retinal Inflammation

Retinal inflammation appears on OCT imaging as hyperreflective foci (HRF) (e.g., Wang, X., et al. (2017) Journal of neuroinflammation 14:121; Matsuda, T., et al. (2017). Investigative ophthalmology & visual science 58, 3254-3261; Vujosevic, S., et al. (2017). Acta ophthalmologica 95, 464-471; Saito, M., et al. (2013). Retina (Philadelphia, Pa.) 33, 559-565; Liu, F., et al. (2016). Molecular vision 22, 352-361; Noma, H., et al. (2010). Journal of inflammation (London, England) 7, 44; Grewal, D. S., et al. (2017). American journal of ophthalmology 177, 116-125).

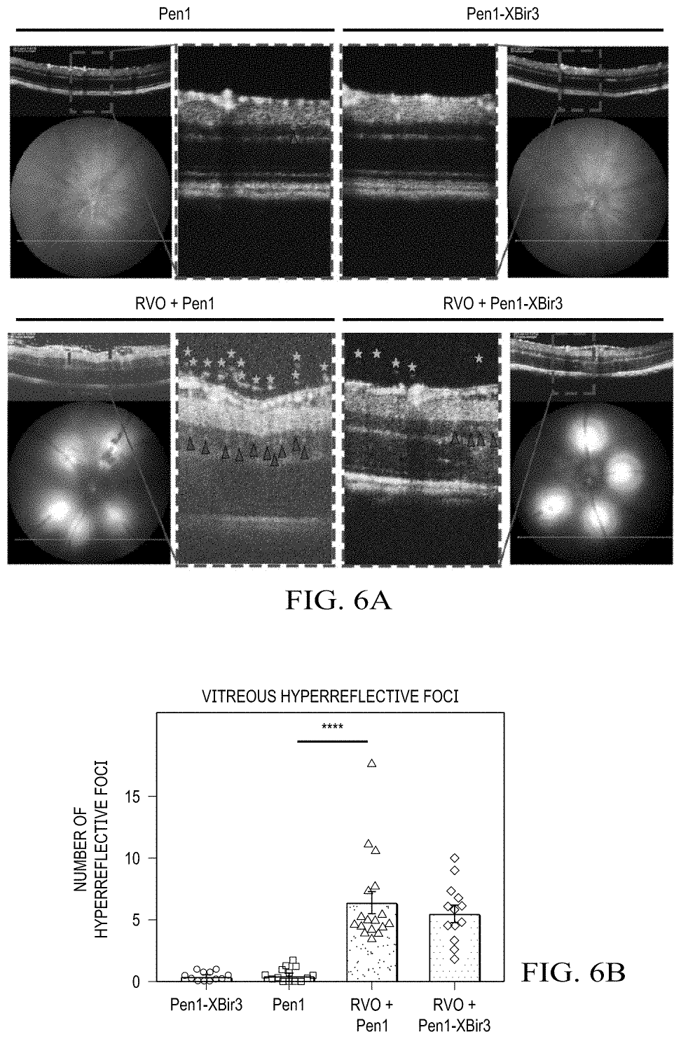

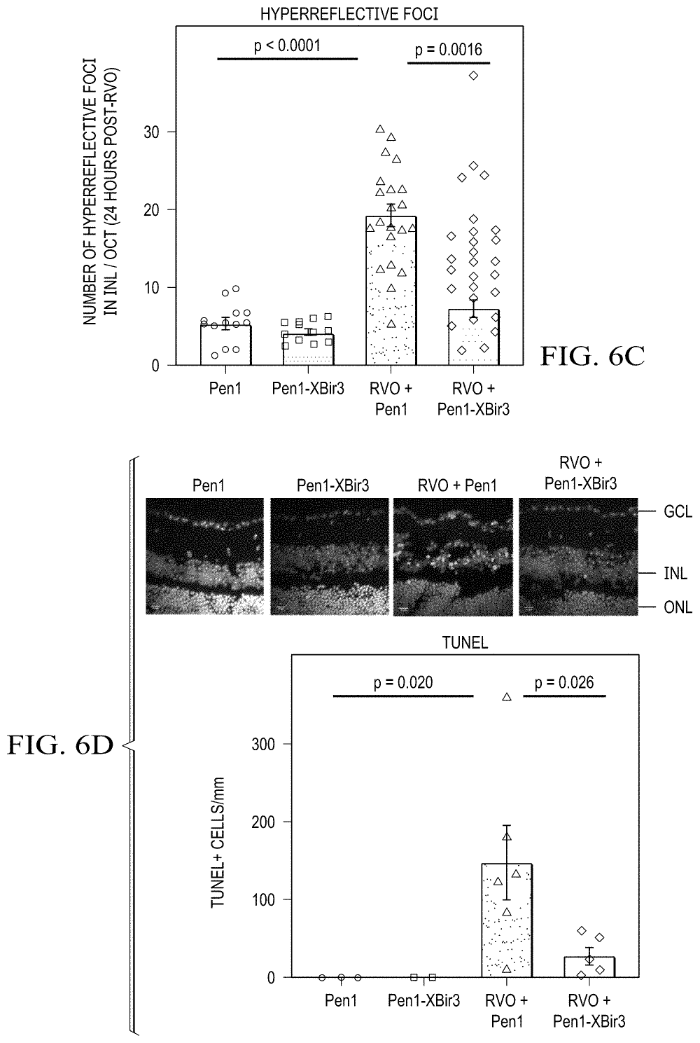

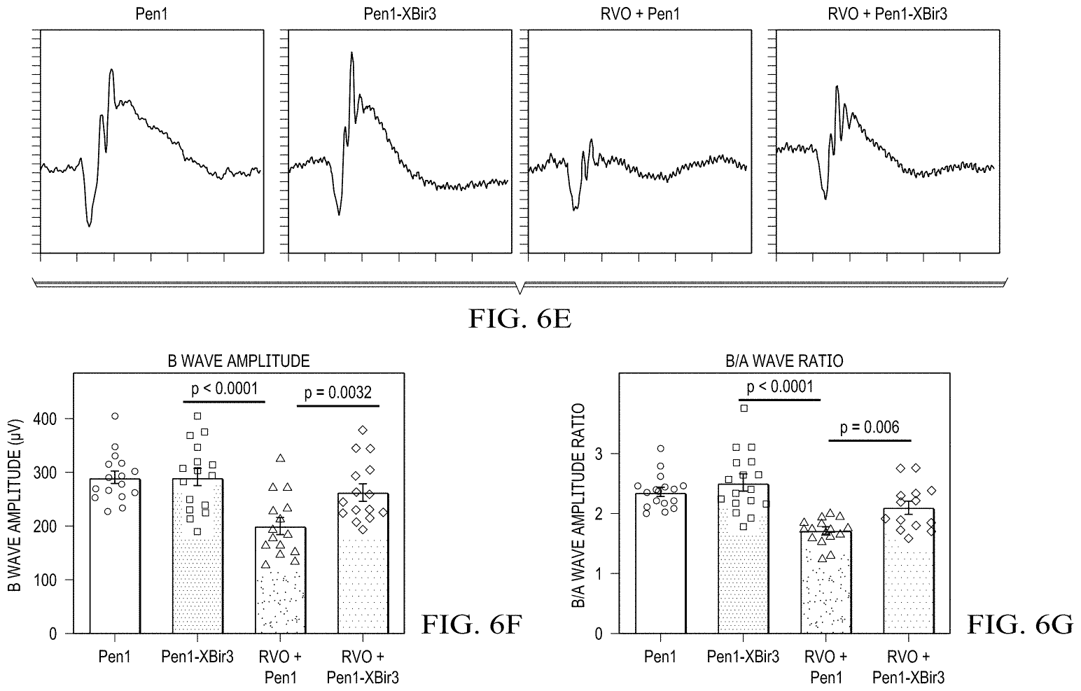

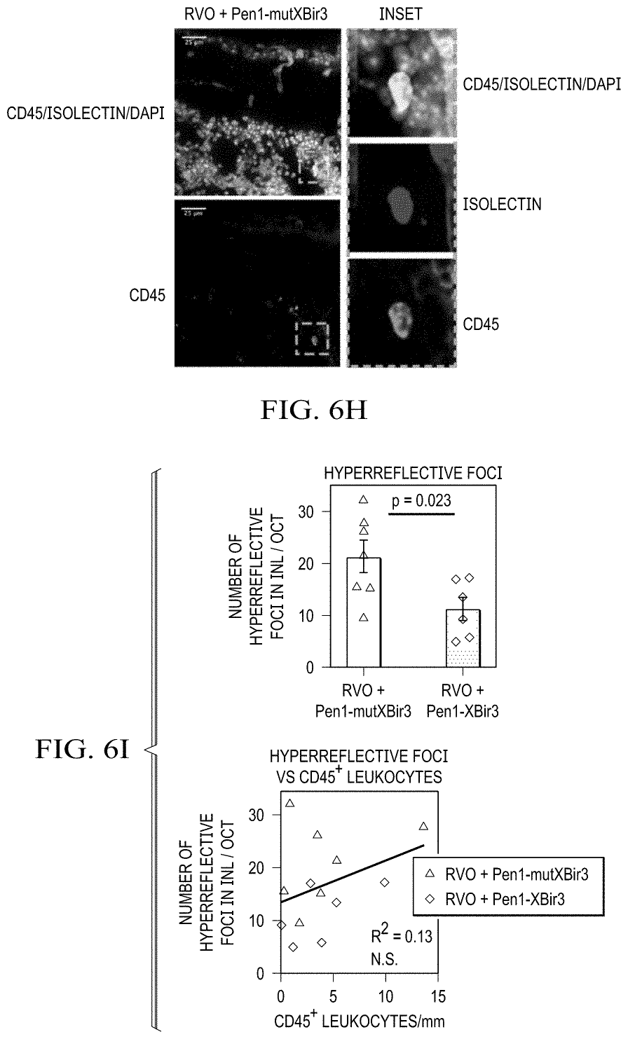

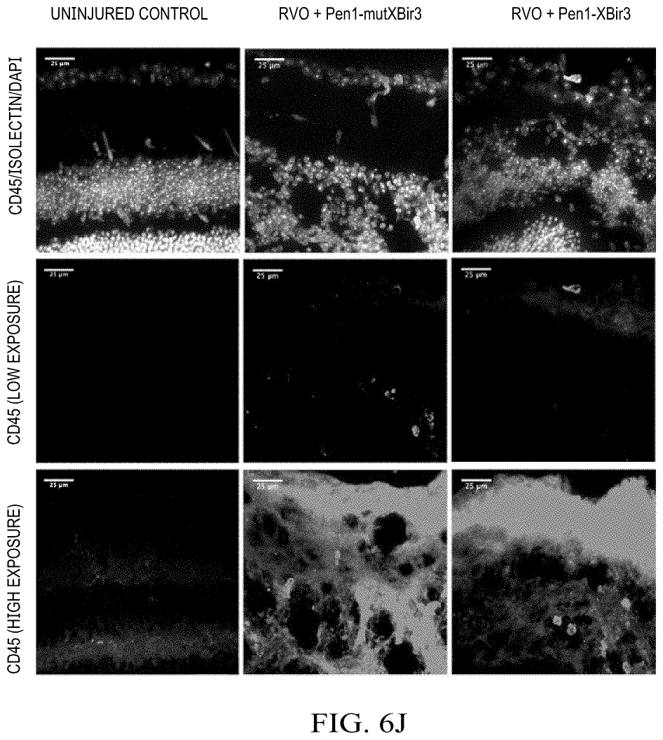

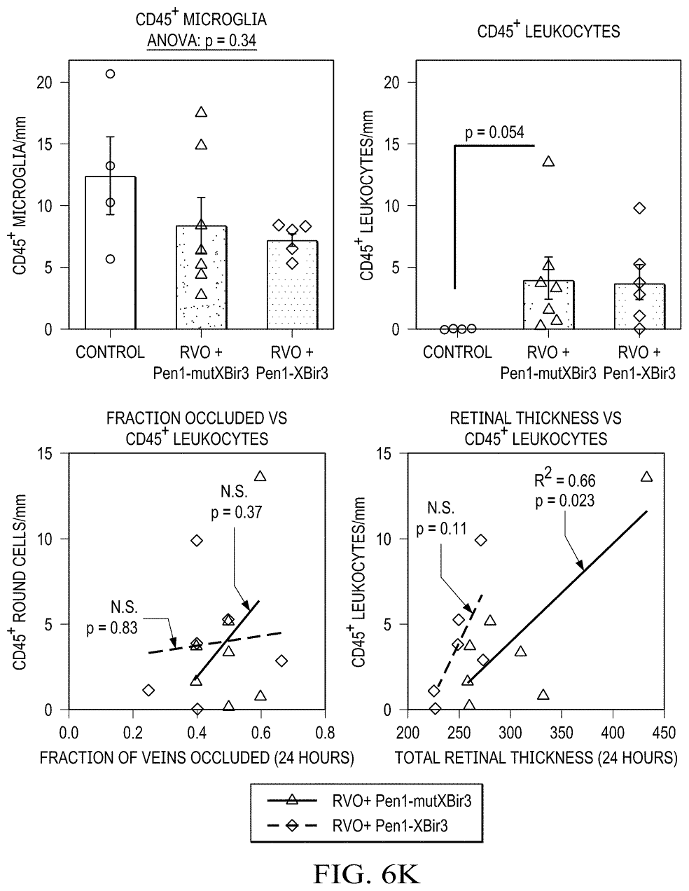

Induction of HRF was observed in the vitreous and retina during the acute edema phase of RVO (FIG. 6A). Eyes which spontaneously reperfused within 24 hrs of RVO did not have a significant increase in retinal HRF (FIG. 10). Caspase-9 inhibition by Pen1-XBir3 did not affect the number of HRF in the vitreous, but reduced the number of HRF in the retina by 50% compared to vehicle (FIG. 6A, FIG. 6B, FIG. 6C). In RVO patients, retinal HRF correlate with disease severity and predict visual outcome after treatment for edema.

To investigate if HRF could be attributed to leukocyte infiltration, immunostaining was performed in Pen1-XBir3 and Pen1-mutXBir3-treated eyes 24 hr post-RVO with CD45 (leukocyte common antigen) and isolectin (FIG. 6H, 6J). CD45+ leukocytes in retinal tissues correlate with retinal swelling 24 hr post-RVO, but not with the fraction of veins occluded (FIG. 6K). The number of leukocytes do not correlate with HRF on OCT imaging, ruling out leukocyte infiltration as a primary source of retinal HRF in RVO (FIG. 6I).

### Example 12. ERG Demonstrates Functional Neuroprotection

Since caspase-9 inhibition by Pen1-XBir3 blocked the activation of caspase-7 in neurons, a TUNEL assay was performed to see if the treatment also blocked cell death. TUNEL staining of retinal sections 24 hrs post-RVO showed fewer dying neurons in retinas that received Pen1-XBir3 (FIG. 6D). The reduction in TUNEL staining is consistent with the protection against retinal atrophy of the INL and ONL, measured by OCT (FIG. 5D).

To test whether the preservation of retinal morphology also conserved retinal function, scotopic focal ERG was performed, 7 days post-RVO. The ERG b wave amplitude, which reflects post-phototransduction activity of inner retinal neurons, is a common readout used in clinical analysis of retinal function. B wave amplitudes are reduced in RVO patients and in mouse models of RVO.

RVO caused a significant reduction in ERG b wave, and a modest reduction in ERG a wave (FIG. 6E). Treatment with Pen1-XBir3 rescued ERG B wave amplitude, and b/a wave ratio, compared to vehicle-treated eyes (FIG. 6F, FIG. 6G). The functional and morphological retinal protection measured by ERG and OCT, respectively, suggest that Pen1-XBir3 may have clinical utility in the treatment of retinal vein occlusion. No harmful effects were detected in uninjured animals treated with Pen1-XBir3, supporting a favorable ocular safety profiles of the treatment.

### Example 13. Cdh5 Cre Drives Inducible Deletion of Caspase-9 Specifically in Endothelial Cells

Since Pen1-XBir3 inhibits caspase-9 activity in all cell types, an inducible endothelial caspase-9 knockout mouse (Casp9iECKO) was generated to interrogate the specific role of endothelial caspase-9 in RVO pathology. The mice were generated by crossing caspase-9 flox/flox (Casp9 WT) animals with an inducible Cre line targeting an endothelial cell promoter (Cdh5PAC-CreERT2) (Pitulescu, M. E., et al. (2010) Nature protocols 5, 1518-1534; Simon, D. J., et al. (2012) The Journal of neuroscience 32, 17540-17553).

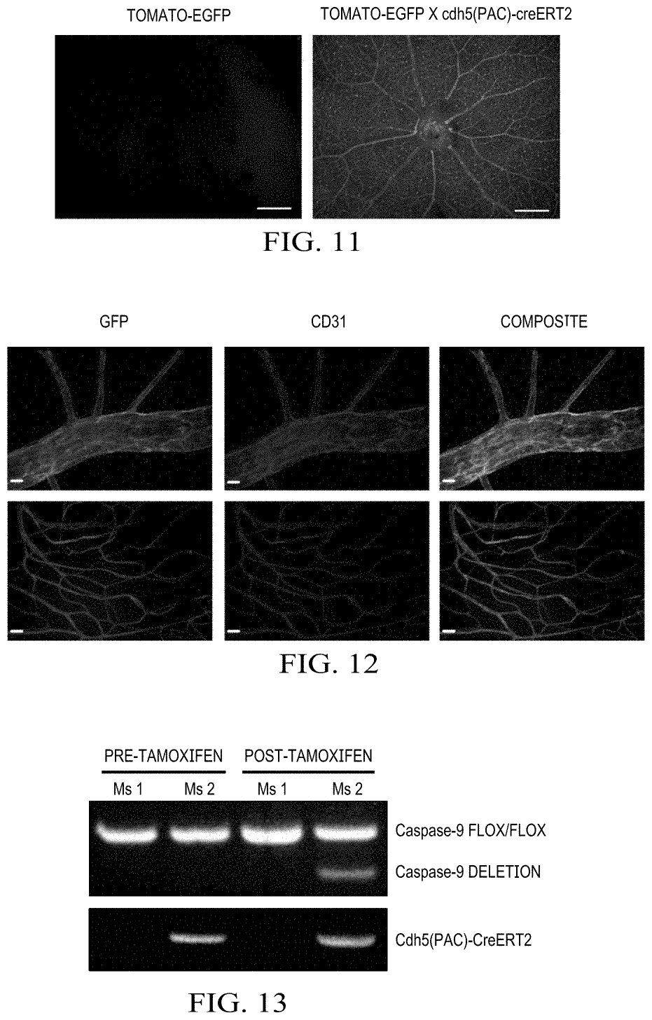

To test the specificity and efficacy of recombination, the inducible endothelial Cre mice was crossed with a Tomato-EGFP mT/mG reporter mouse (Muzumdar, M. D. et al. (2007). Genesis 45, 593-605). Tamoxifen treatment induced recombination specifically in the vasculature, as visualized by GFP staining of retinal flatmounts 2 weeks after tamoxifen treatment in 6 week old mice (FIG. 11, FIG. 12). PCR genotyping confirmed tamoxifen induction of caspase-9 recombination in tissue digests from Casp9iECKO animals (FIG. 13). Immunostaining showed no activation of caspase-7 in blood vessels of Casp9iECKO mice, confirming functional endothelial caspase-9 deficiency (FIG. 14).

### Example 14. Inducible Endothelial Cell Deletion of Caspase-9 Does Not Alter Basal Retinal Function

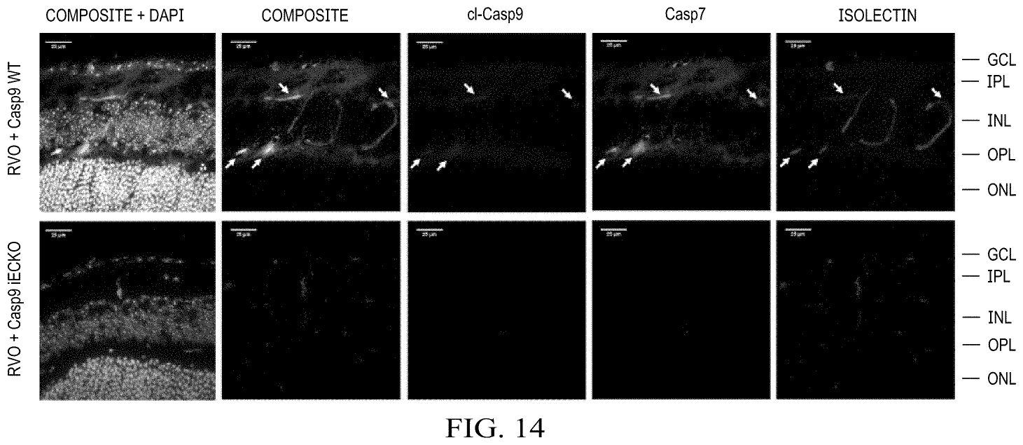

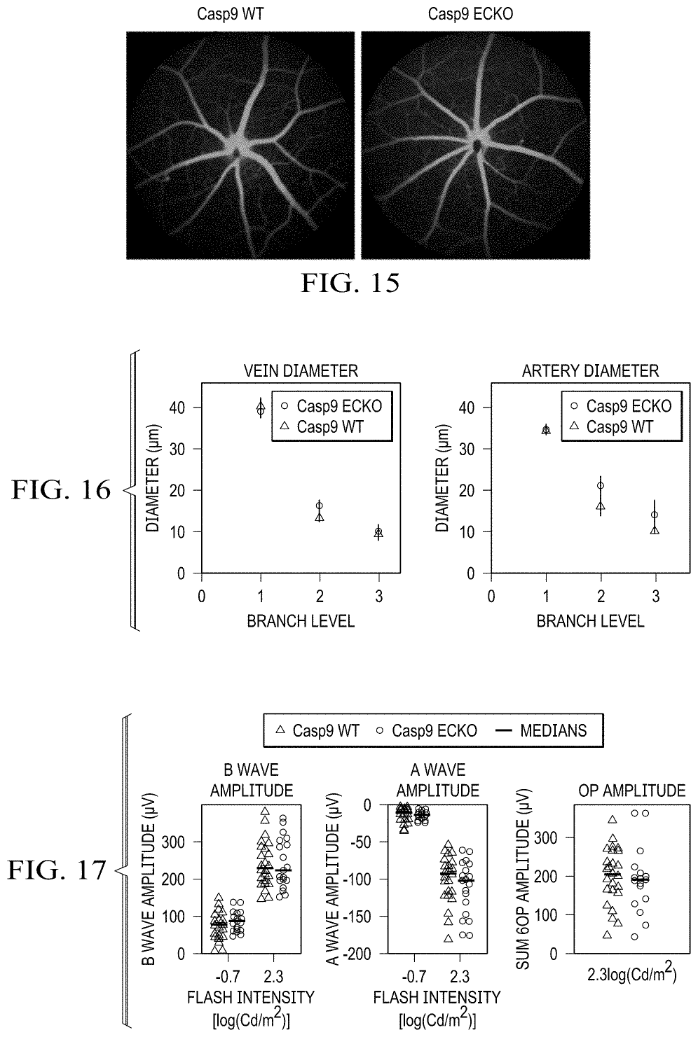

By inducing recombination in mature animals, potential complications of disrupting physiologic endothelial caspase-9 activity during development were avoided. There were no differences in baseline retinal morphology between Casp9iECKO animals and their littermate controls (FIG. 15, FIG. 16). Fluorescein angiography showed normal vascular development and no defects in vascular permeability, vessel density or diameter. ERG analysis of retinal function shows equivalent amplitudes and timing of a wave, b wave, and oscillatory potentials in Casp9 WT and Casp9iECKO animals (FIG. 17).

### Example 15. Deletion of Endothelial Caspase-9 is Sufficient for Morphological and Functional Retinal Protection from RVO

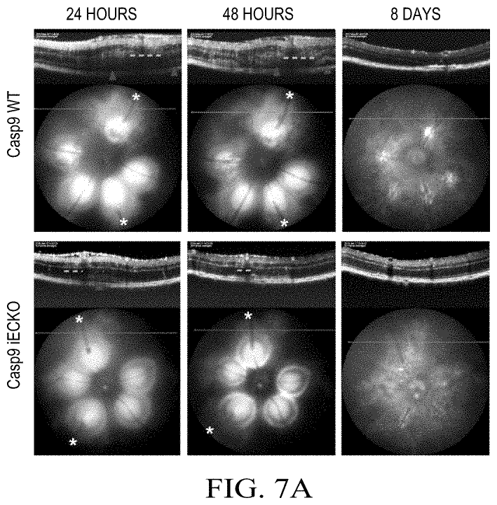

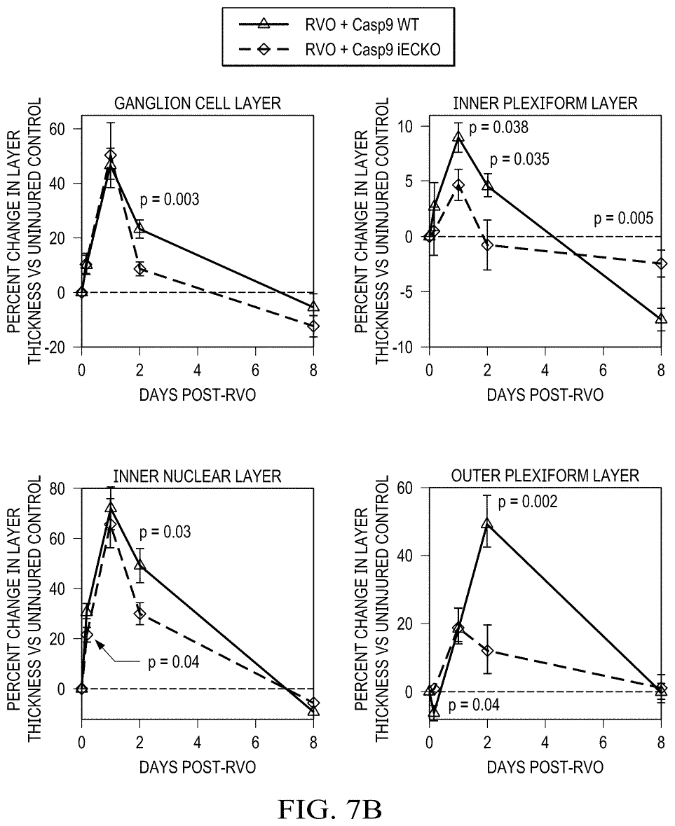

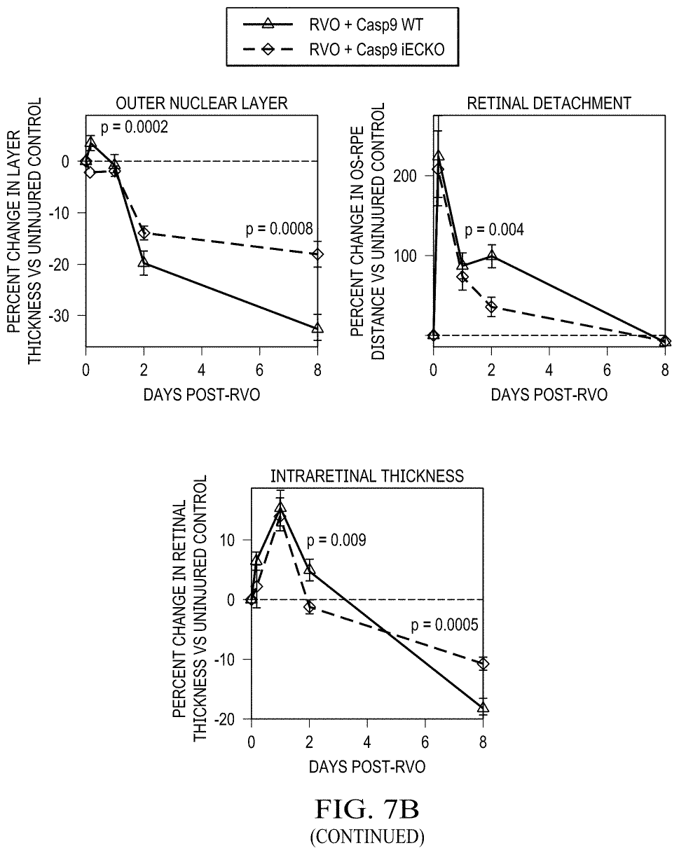

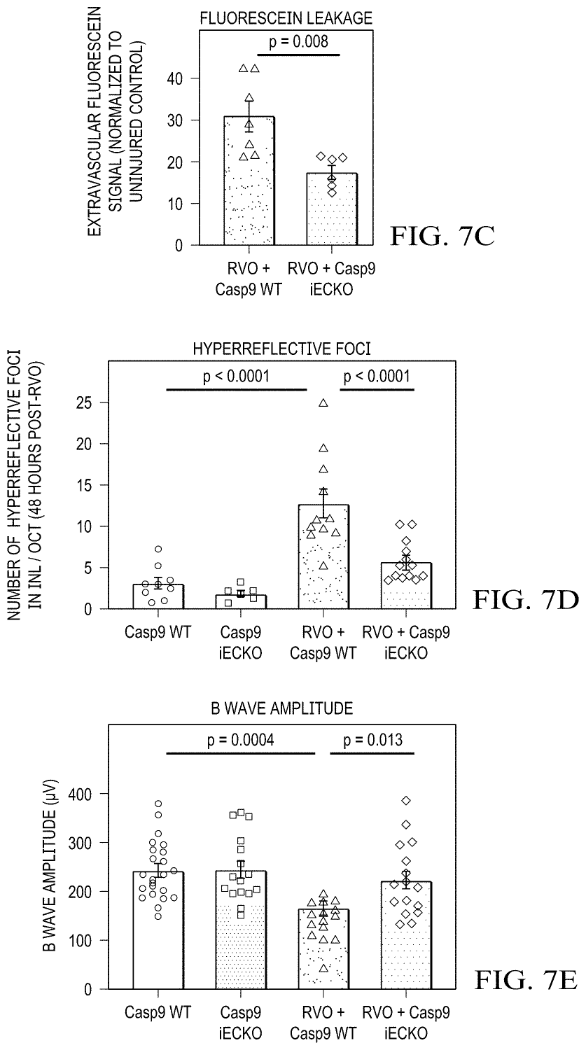

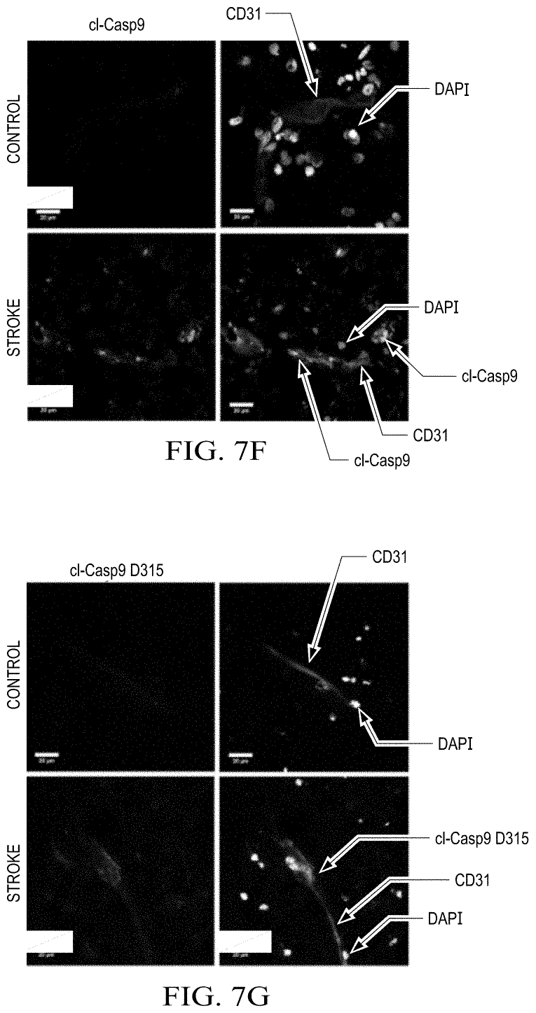

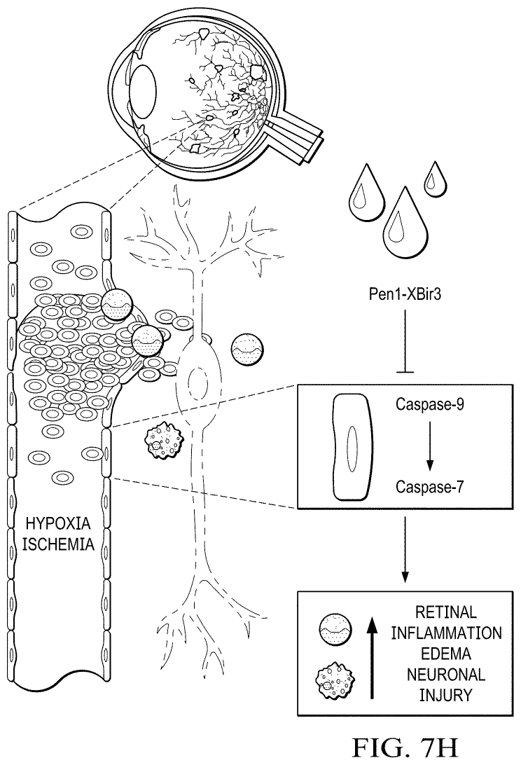

RVO was induced in Casp9iECKO mice and Casp9 WT littermate controls, and retinal injury was assessed by fluorescein angiography, OCT imaging, and ERG. Casp9iECKO mice develop less fluorescein leakage and less retinal swelling than Casp9 WT animals (FIG. 7A, FIG. 7B, FIG. 7C). Casp9iECKO mice have less retinal detachment, and less retinal thinning (FIG. 7B). Quantification of retinal HRF showed a 50% reduction in inflammation in Casp9iECKO animals (FIG. 7D). ERG assessment of retinal function in Casp9iECKO mice phenocopied the results from caspase-9 inhibition by Pen1-XBir3. At 1 week after RVO, Casp9 WT mice had a significant decrease in b wave amplitude, which was rescued in Casp9iECKO animals (FIG. 7E). These results indicate that endothelial caspase-9 specifically drives vascular dysfunction following RVO, and that targeting caspase signaling in endothelial cells can attenuate retinal inflammation and neuronal injury.

### Example 16. Autocleaved Caspase-9 Detected in Endothelial Cells from Ischemic Human Brain Tissue

Having established therapeutic potential of caspase-9 inhibition in mouse models of ischemic injury, it was assessed whether endothelial caspase-9 activation is present in ischemic CNS tissues from human patients. The retina is a part of the CNS, and RVO shares similar molecular pathology to ischemic stroke (D'Onofrio, P. M., and Koeberle, P. D. (2013). Acta pharmacologica Sinica 34, 91-103). Brain sections from patients who died after cerebral infarction were examined; cleaved caspase-9 was found in blood vessels in the patient brain sections but not in the age-matched controls (FIG. 7F, FIG. 7G). Autocleavage of human caspase-9 produces the D315 epitope, which is a target of XBir3 (Denault, J. B., et al. (2007) The Biochemical journal 405, 11-19). Autocleaved caspase-9 (D315) was found to co-localize with the endothelial marker CD31 (FIG. 7G), closely resembling the staining seen in mouse RVO samples (FIG. 2) suggesting endothelial activation of caspase-9 is preserved in both mouse and human neurovascular injury.

### Example 17: Materials and Methods

The following materials and methods were used, as applicable, in the above Examples 1-16.

Animals

Wild type C57BL/6 2 month old mice were purchased from Jackson Laboratories (Maine, US). Endothelial specific caspase-9 inducible knockout (casp9iECKO) mice were bred by crossing Caspase-9 flox/flox mice (described in D. J. Simon et al., A caspase cascade regulating developmental axon degeneration. The Journal of neuroscience: the official journal of the Society for Neuroscience 32, 17540-17553 (2012)) and endothelial cell-CreERT2 mice (Cdh5(PAC)-CreERT2) (described in M. E. Pitulescu, I. Schmidt, R. Benedito, R. H. Adams, Inducible gene targeting in the neonatal vasculature and analysis of retinal angiogenesis in mice. Nature protocols 5, 1518-1534 (2010); K. Gaengel et al., The sphingosine-1-phosphate receptor S1PR1 restricts sprouting angiogenesis by regulating the interplay between VE-cadherin and VEGFR2. Developmental cell 23, 587-599 (2012)). Cre reporter (as described in M. D. Muzumdar, B. Tasic, K. Miyamichi, L. Li, L. Luo, A global double-fluorescent Cre reporter mouse. Genesis (New York, N.Y.: 2000) 45, 593-605 (2007)) was used mT/mG mice. All mice were on a C57/B16 background. Recombination was induced in 6 week old animals by intraperitoneal (IP) injection with 2 mg tamoxifen for 5 consecutive days.

An ocular distribution study of Pen1-XBir3 eye drops was performed by EyeCRO (Oklahoma City, Okla.). Adult female New Zealand White Rabbits (N=4) had plasma collected and then received bilateral topical administration of 200 μg Pen1-XBir3, twice daily for a period of 4.5 consecutive days. On day 5, 4 hours after administration of the final dose of Pen1-XBir3, plasma was collected from the animals, and the eyes were enucleated and the retinas dissected.

All investigations were performed in accordance with the ARVO statement for the Use of Animals in Ophthalmic and Vision Research, and the experiments were approved and monitored by the Institutional Animal Care and Use Committee of Columbia University.

### Human Brain Tissue

Columbia University's Brain Bank provided human brain samples of postmortem tissue from two patients who died following ischemic stroke and two control patients without diagnostic abnormalities, which were previously described in N. Akpan et al., Intranasal delivery of caspase-9 inhibitor reduces caspase-6-dependent axon/neuron loss and improves neurological function after stroke. The Journal of Neuroscience: the official journal of the Society for Neuroscience 31, 8894-8904 (2011).

### RVO Model

RVO was performed as described in A. Ebneter, C. Agca, C. Dysli, M. S. Zinkernagel, Investigation of retinal morphology alterations using spectral domain optical coherence tomography in a mouse model of retinal branch and central retinal vein occlusion. PloS one 10, e0119046 (2015), and S. Fuma et al., A pharmacological approach in newly established retinal vein occlusion model. Scientific reports 7, 43509 (2017).

Mice received 0.75 mg rose bengal by tail vein injection. For all imaging and RVO procedures, mice were anesthetized with ketamine (80-100 mg/kg) and xylazine (5-10 mg/kg). Eyes were dilated with tropicamide and phenylephrine chloride eye drops. Major veins in each eye were ablated using Micron IV image guided laser (532 nm) (Phoenix Research Labs, Pleasanton, Calif., USA). Three adjacent laser pulses (power 100 mW, spot size 50 M, duration 1 second) were delivered to each vein an average distance of 2 disc diameters from the optic nerve center. Sham RVO was performed using identical parameters as the RVO procedure, with three adjacent laser bums targeted to the capillary bed between each of the major veins. RVO was judged successful if a vein occlusion was observed by fundus imaging at 24 hours post-RVO.

### Fluorescein Angiography

Fluorescein angiography images were captured 24 hr, 48 hr, and 8 days post-RVO using a Micron IV fundus camera (Phoenix Research Labs) using maximum illumination intensity. Fluorescein visualization of vascular morphology in Casp9iECKO and Casp9 WT animals was performed 10 min after intraperitoneal injection with 1% fluorescein. Fluorescein imaging of edema was performed 5 min after IP injection of 1% fluorescein. Fluorescein leakage was analyzed using Image J (Rueden, C. T.; Schindelin, J. & Hiner, M. C. et al. (2017), “ImageJ2: ImageJ for the next generation of scientific image data”, BMC Bioinformatics 18:529, doi:10.1186/s12859-017-1934-z) by measuring the mean fluorescein signal in the retinal tissue around an occluded vein. To control for mouse-to-mouse variability of fluorescein signal intensity, the signal was normalized against the mean signal intensity of fluorescein within major arteries adjacent to the area measured.

### Image Guided Optical Coherence Tomography

OCT images were captured using a Phoenix Micron IV image-guided OCT system (Phoenix Research Labs). For each eye, 2 vertical and 2 horizontal OCT scans were captured approximately 75 μm distal from the periphery of the RVO burn areas. InSight software (Phoenix Research Labs, Pleasanton, Calif.) was used to generate segmentation of the individual retinal layers in each OCT scan, which were then analyzed to calculate average layer thicknesses. For each eye, the four OCT images were averaged to generate mean retinal thickness values. Change in retinal thickness for injured eyes was measured against composite averages from uninjured controls. Hyperreflective foci were analyzed from 24 hr and 48 hr post-RVO OCT images using Image J. OCT images were processed using the Image J Despeckle function. A threshold was applied to the INL (for quantification of retinal HRF) or vitreous (for quantification of vitreal HRF), selecting hyperreflective regions based on pixels having two standard deviations above the mode pixel intensity for that layer. The number of hyperreflective foci was quantified using the Image J Analyze Particles function.

### Focal Electroretinography (ERG)

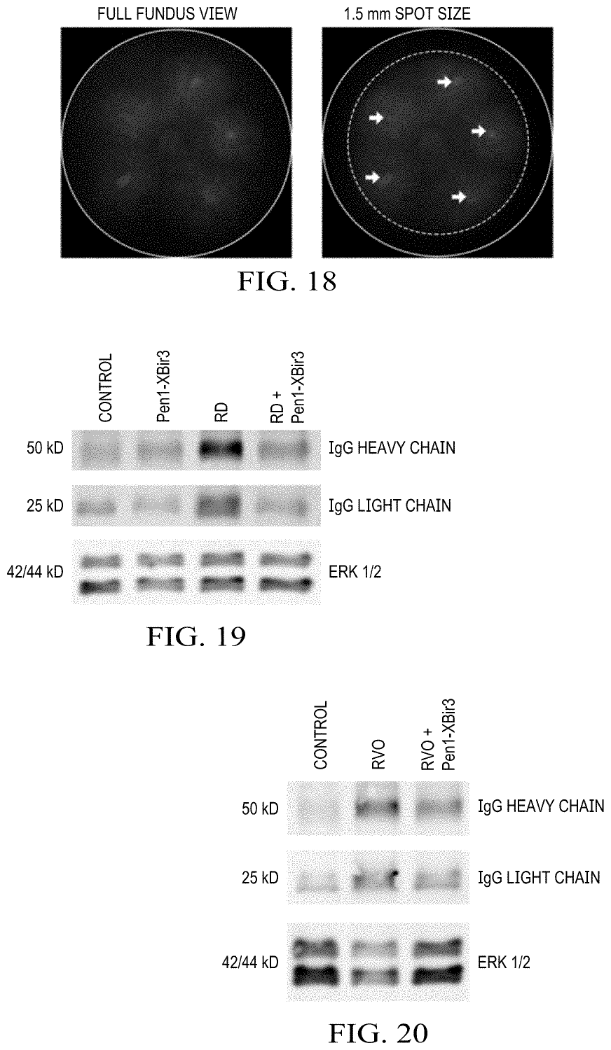

Electroretinograms were recorded 7 days post-RVO using the Micron IV Image-Guided Focal ERG system (Phoenix Research Labs) on dark-adapted mice. Focal ERG was recorded using a flash spot size of 1.5 mm, centered on the optic nerve head (FIG. 18), and white light LED stimulus intensity of −0.7 log(Cd sec/m2) and 2.3 log(Cd sec/m2). The amplitude of the A-wave was measured from the baseline to the maximum A-wave peak, and the B wave was measured from the maximum A-wave peak to the maximum B wave peak. Oscillatory potentials (OP) were derived using a 30 Hz-300 Hz filter, and the sum of the first 6 OP was used to calculate sum OP amplitude.

### Immunohistochemistry and Antibodies

For immunohistochemistry and Western blotting, anti-caspase-8 (Cell Signaling Technology, Danvers, Mass., US, 4790S), and-CD11b (eBioscience, Thermo-Fisher Scientific, Waltham, Mass., US), anti-GFAP (Thermo Fisher), anti-full-length caspase-9 (Abcam ab28131) and anti-cleaved caspase-9 (Abcam ab2325), anti-D315 human cleaved caspase-9 (Cell Signaling), anti-cleaved caspase-3 (Cell Signaling), anti-cleaved caspase-7 (MBL International Corp. Woburn, Mass., US), anti-PH2AX (Cell Signaling), anti-CD31 (BD Biosciences, San Jose, Calif., US), anti-NeuN (EMD Millipore, Burlington, Mass. US), and anti-GFP (Thermo Fisher Scientific) were used. For Western blotting, anti-His (GenScript U.S.A. Piscataway, N.J., US), anti-XIAP (Cell Signaling), anti-VEGF (Abcam, Cambridge, UK) and anti-ERK (Santa Cruz Biotechnology, Dallas, Tex., US) were used. TUNEL assay was performed using the DeadEnd Fluorometric TUNEL system (Promega Corp., Madison, Wis., US). Mice were euthanized with Ketamine 80-100 mg/kg plus Xylazine 5-10 mg/kg and perfused followed by fixation with 4% paraformaldehyde. Retinal flatmounts were permeabilized for 2 hr at RT in PBS with 1% Triton X-100, prior to blocking step. Retinal flatmounts and sections (thickness 10 μm) were blocked with 10% normal goat serum/1% bovine serum albumin (BSA) with 0.1% Triton X-100 in phosphate buffered saline (PBS), incubated with primary antibody overnight at 4° C., washed with PBS, and incubated with the species-appropriate Alexa Fluor-conjugated secondary antibody (Invitrogen, Carlsbad, Calif., US) for 2 hr at room temperature. Human samples were additionally treated with Sudan Black (1% in 70% EtOH) to minimize autofluorescence. Microscopy was captured using a Zeiss LSM 800 confocal microscope (Karl Zeiss AG, Oberkochen, Germany), and the images processed in FIJI using Adjust Brightness/contrast (Schindelin, J.; Arganda-Carreras, I. & Frise, E. et al. (2012), “Fiji: an open-source platform for biological-image analysis”, Nature methods 9(7): 676-682, PMID 22743772, doi:10.1038/nmeth.2019). Antibodies with high nonspecific background were denoised using Noise>Remove outliers.

### Western Blot Analysis

Western blots were blocked for 1 hr with 5% BSA in TBS-Tween (0.05%), incubated with primary antibody overnight at 4° C., washed with TBS-Tween, and incubated with the species-appropriate secondary antibodies for 2 hr at room temperature. Western blots were imaged using an Odyssey system (LI-COR Biosciences, Lincoln, Nebr., US). Western blots were quantified using Image Studio Lite software (LI-COR Biosciences).

### Pen1-XBir3

XBir3 was purified as described in C. Sun et al., NMR structure and mutagenesis of the third Bir domain of the inhibitor of apoptosis protein XIAP. The Journal of biological chemistry 275, 33777-33781 (2000). Pen1 (PolyPeptide Group, Torrence, Calif., US) was mixed at a 1:2 molar ratio with purified XBir3 and incubated for 2 h at 37° C. to generate disulfide-linked Pen1-XBir3. Linkage was assessed by 20% SDS-PAGE and Western blotting with anti-His antibody. Eye drops containing 10 μg Pen1-XBir3 were administered immediately following RVO, and again at 24 hr. An equivalent volume of saline containing unlinked Pen1 was administered as a vehicle control.

### Quantification and Statistical Analysis

In RVO studies, excessive damage, characterized by complete retinal detachment within 48 hr hours post-RVO, or intravitreal hemorrhaging that obscured view of the retina, occurred in 5-10% of eyes, which were excluded by a blinded observer from all subsequent analyses. Eyes were included for subsequent analysis if fundus imaging identified occlusion of at least 1 vein 24 hours post-RVO, and OCT analysis of retinal layer thickness was within 2.5 standard deviations of the mean for each treatment group. Data were analyzed using Excel (Microsoft, Wash., US) and GraphPad (GraphPad Software, Inc. LaJolla, Calif.) statistical software. Statistical tests, n values and p values are all located in the figures and/or legends. Significance was defined as p<0.05.

### Example 18: Targeting Caspase-9 Reduced Immunoglobin Levels in a Retinal Detachment (RD) Model of Retinal Injury

In addition to the retinal vein occlusion model of retinal injury described herein, another model of retinal injury is the retinal detachment (RD) model. Retinal detachment can be induced by intravetrial injection of hypertonic phosphate-buffered saline (PBS).

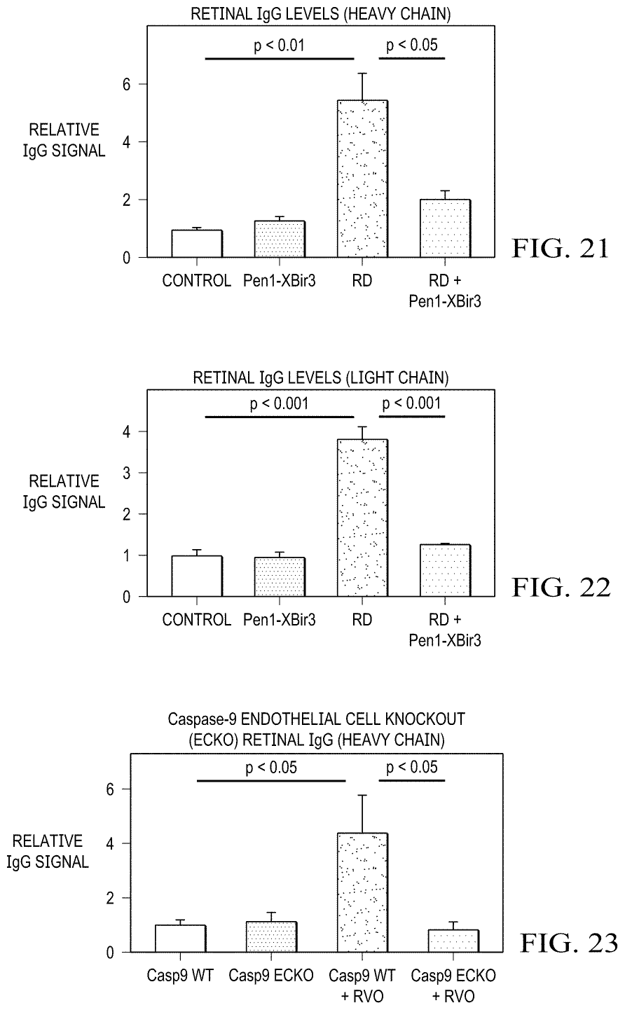

In this Example, RD was induced by intravitreal injection of 5 uL of 10×PBS. Retinal lysates from untreated wildtype mice (control), Pen1-XBir3-treated wildtype mice (Pen1-XBir3), untreated wildtype mice 24 hr after induction of retinal detachment (RD), and Pen1-XBir3-treated wildtype mice 24 hr after induction of retinal detachment (RD+Pen1-XBir3) were analyzed by Western blot of mouse IgG. The Western blot membrane was probed for mouse immunoglobulin (IgG) and ERK1/2 loading control. RD induces 5-fold increase of IgG, which is abrogated by treatment with Pen1-XBir3. (FIG. 19). FIG. 21 is a graph reporting exemplary quantification of IgG heavy chain from FIG. 19.

FIG. 22 is a graph reporting exemplary quantification of IgG light chain from FIG. 19.

### Example 19: Targeting Caspase-9 Reduced Immunoglobin Levels in an RVO Model of Retinal Injury

Retinal lysates from untreated wildtype mice (control), untreated wildtype mice 24 hr after induction of retinal vein occlusion (RVO), and Pen1-XBir3-treated wildtype mice 24 hr after induction of retinal vein occlusion (RVO+Pen1-XBir3) were analyzed by Western blot of mouse IgG. The Western blot was probed for IgG and ERK1/2 loading control. Increase in IgG after RVO is abrogated by treatment with Pen1-XBir3 (FIG. 20).

### Example 20: Casp9iECKO Mice have Lower Immunoglobin Levels Compared to WT Mice Following RVO

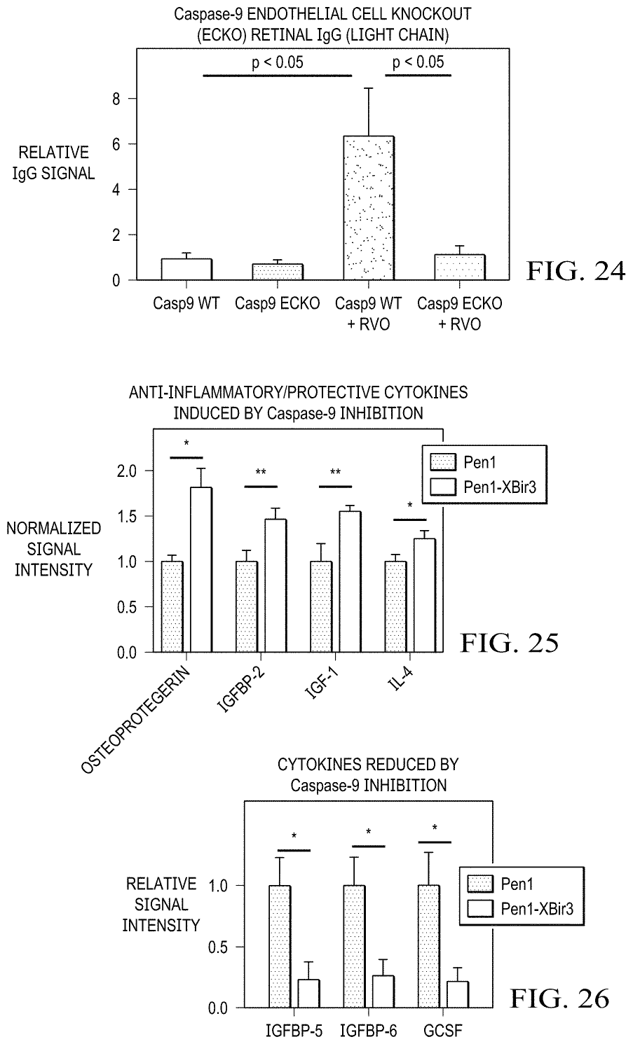

Retinal lysates were collected from intact retinas, and 24 hr post-RVO from endothelial caspase-9 knockout mice (Casp9iECKO) and caspase-9 wildtype littermate controls (Casp9 WT) and immunoglobulin levels were analyzed by western blot. Levels of IgG heavy chain (FIG. 23) and IgG light chain (FIG. 24) in retinal lysates were quantified. Immunoglobulin levels in RVO-treated Casp9 WT mice were increased compared to untreated Casp9 WT mice and untreated Casp9iECKO mice. In contrast, RVO-treated Casp9iECKO showed similar immunoglobulin levels to untreated Casp9 WT mice and untreated Casp9iECKO mice.

### Example 21: Pen1-XBir3 Treatment Effect on Cytokine Levels in Healthy Mice

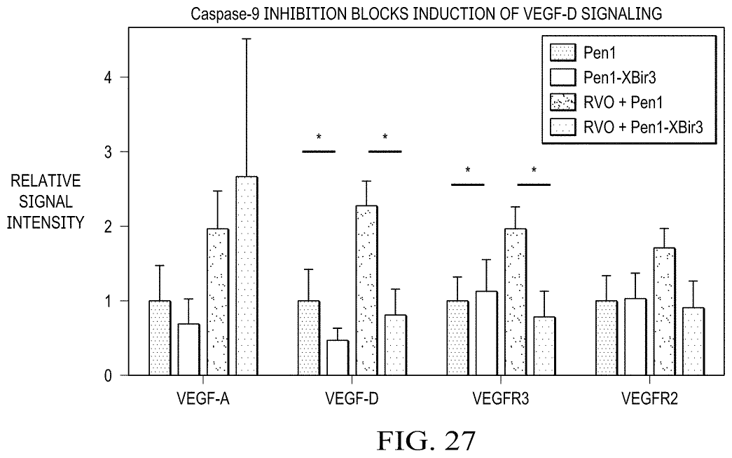

Healthy adult C57/B16J mice were treated with eye-drops containing either Pen1-Saline or 10 μg Pen1-XBir3. Retinas were harvested 24 hours after treatment, lysed, and protein levels analyzed by antibody array. Levels of anti-inflammatory/protective cytokines including osteoprotegerin, Insulin-Like Growth Factor Binding Protein 2 (IGFBP-2), Insulin-Like Growth Factor-1 (IGF-1) and interleukin-4 (IL-4) were increased by caspase-9 inhibition following Pen1-XBir3 treatment (FIG. 25). In contrast, levels of Insulin-like growth factor-binding protein-5 (IGFBP-5), Insulin-like growth factor-binding protein-6 (IGFBP-5) and granulocyte colony-stimulating factor (GCSF) were decreased following Pen1-XBir3 treatment (FIG. 26). Student's T-test *p<0.05, **p<0.01, n=7. Without limitation to theory, these data show that caspase-9 inhibition can modulate inflammatory signaling in the absence of apoptosis, or hypoxia/ischemia injury.

### Example 22: Pen1-XBir3 Treatment Effect on VEGF Signaling Pathway in Mice Following RVO

RVO was induced in healthy adult C57/B16J mice. Animals were treated with eye-drops containing either Pen1-Saline or 10 μg Pen1-XBir3 immediately following induction of RVO or in mice without RVO. Retinas were harvested 24 hours after treatment, lysed, and protein levels analyzed by antibody array. Caspase-9 inhibition blocked induction of VEGF-D signaling in RVO treated mice (FIG. 27). ANOVA *p<0.05, **p<0.01 n=6-7.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

Patents, patent applications, publications, procedures, and the like are cited throughout this application and below, the disclosures of which are incorporated herein by reference in their entireties and specifically as they relate to materials and processes expressly mentioned herein.

A. London, I. Benhar, M. Schwartz, The retina as a window to the brain-from eye research to CNS disorders. Nature reviews. Neurology 9, 44-53 (2013).

S. T. Hou, J. P. MacManus, Molecular mechanisms of cerebral ischemia-induced neuronal death. Int Rev Cytol 221, 93-148 (2002).

J. L. Yang, S. Mukda, S. D. Chen, Diverse roles of mitochondria in ischemic stroke. Redox Biol 16, 263-275 (2018).

C. Kaur, W. S. Foulds, E. A. Ling, Blood-retinal barrier in hypoxic ischaemic conditions: basic concepts, clinical features and management. Progress in retinal and eye research 27, 622-647 (2008).

J. P. Ehlers et al., Therapies for Macular Edema Associated with Branch Retinal Vein Occlusion: A Report by the American Academy of Ophthalmology. Ophthalmology 124, 1412-1423 (2017).

M. Ho, D. T. Liu, D. S. Lam, J. B. Jonas, RETINAL VEIN OCCLUSIONS, FROM BASICS TO THE LATEST TREATMENT. Retina (Philadelphia, Pa.) 36, 432-448 (2016).

P. A. Campochiaro et al., Long-term outcomes in patients with retinal vein occlusion treated with ranibizumab: the RETAIN study. Ophthalmology 121, 209-219 (2014).

A. Ebneter, C. Agca, C. Dysli, M. S. Zinkernagel, Investigation of retinal morphology alterations using spectral domain optical coherence tomography in a mouse model of retinal branch and central retinal vein occlusion. PloS one 10, e0119046 (2015).

S. Fuma et al., A pharmacological approach in newly established retinal vein occlusion model. Scientific reports 7, 43509 (2017).

H. Zhang et al., Development of a new mouse model of branch retinal vein occlusion and retinal neovascularization. Japanese journal of ophthalmology 51, 251-257 (2007).

A. Jaulim, B. Ahmed, T. Khanam, I. P. Chatziralli, Branch retinal vein occlusion: epidemiology, pathogenesis, risk factors, clinical features, diagnosis, and complications. An

I. Klaassen, C. J. Van Noorden, R. O. Schlingemann, Molecular basis of the inner blood-retinal barrier and its breakdown in diabetic macular edema and other pathological conditions. Progress in retinal and eye research 34, 19-48 (2013).

N. Akpan et al., Intranasal delivery of caspase-9 inhibitor reduces caspase-6-dependent axon/neuron loss and improves neurological function after stroke. The Journal of neuroscience : the official journal of the Society for Neuroscience 31, 8894-8904 (2011).

R. J. Blanch et al., Caspase-9 mediates photoreceptor death after blunt ocular trauma. Investigative ophthalmology & visual science 55, 6350-6357 (2014).

J. B. Denault, B. P. Eckelman, H. Shin, C. Pop, G. S. Salvesen, Caspase 3 attenuates XIAP (X-linked inhibitor of apoptosis protein)-mediated inhibition of caspase 9. The Biochemical journal 405, 11-19 (2007).

S. Chen et al., The effect of concentration and duration of normobaric oxygen in reducing caspase-3 and -9 expression in a rat-model of focal cerebral ischaemia. Brain research 1618, 205-211 (2015).

P. Garnier et al., Hypoxia induces caspase-9 and caspase-3 activation without neuronal death in gerbil brains. The European journal of neuroscience 20, 937-946 (2004).

N. Ferrara, H. P. Gerber, J. LeCouter, The biology of VEGF and its receptors. Nature medicine 9, 669-676 (2003).

J. D. Hood, C. J. Meininger, M. Ziche, H. J. Granger, VEGF upregulates ecNOS message, protein, and NO production in human endothelial cells. The American journal of physiology 274, H1054-1058 (1998).

D. S. Grewal, M. L. O'Sullivan, M. Kron, G. J. Jaffe, Association of Disorganization of Retinal Inner Layers With Visual Acuity In Eyes With Uveitic Cystoid Macular Edema. American journal of ophthalmology 177, 116-125 (2017).

J. W. Kang, H. Lee, H. Chung, H. C. Kim, Correlation between optical coherence tomographic hyperreflective foci and visual outcomes after intravitreal bevacizumab for macular edema in branch retinal vein occlusion. Graefe's archive for clinical and experimental ophthalmology=Albrecht von Graefes Archiv fur klinische and experimentelle Ophthalmologie 252, 1413-1421 (2014).

W. C. Ou, D. M. Brown, J. F. Payne, C. C. Wykoff, Relationship Between Visual Acuity and Retinal Thickness During Anti-Vascular Endothelial Growth Factor Therapy for Retinal Diseases. American journal of ophthalmology 180, 8-17 (2017).

S. Vujosevic et al., Imaging retinal inflammatory biomarkers after intravitreal steroid and anti-VEGF treatment in diabetic macular oedema. Acta ophthalmologica 95, 464-471 (2017).

R. L. Wong, J. W. Lee, G. S. Yau, I. Y. Wong, Relationship between Outer Retinal Layers Thickness and Visual Acuity in Diabetic Macular Edema. BioMed research international 2015, 981471 (2015).

L. Yang, D. Bula, J. G. Arroyo, D. F. Chen, Preventing retinal detachment-associated photoreceptor cell loss in Bax-deficient mice. Investigative ophthalmology & visual science 45, 648-654 (2004).

K. Nishijima et al., Vascular endothelial growth factor-A is a survival factor for retinal neurons and a critical neuroprotectant during the adaptive response to ischemic injury. The American journal of pathology 171, 53-67 (2007).

A. Ebneter, D. Kokona, N. Schneider, M. S. Zinkernagel, Microglia Activation and Recruitment of Circulating Macrophages During Ischemic Experimental Branch Retinal Vein Occlusion. Investigative ophthalmology & visual science 58, 944-953 (2017).

D. Kokona, N. U. Haner, A. Ebneter, M. S. Zinkernagel, Imaging of macrophage dynamics with optical coherence tomography in anterior ischemic optic neuropathy. Experimental eye research 154, 159-167 (2017).

T. Matsuda et al., Vascular Adhesion Protein-1 Blockade Suppresses Ocular Inflammation After Retinal Laser Photocoagulation in Mice. Investigative ophthalmology & visual science 58, 3254-3261 (2017).

X. Wang et al., Rapid monocyte infiltration following retinal detachment is dependent on non-canonical IL6 signaling through gp130. Journal of neuroinflammation 14, 121 (2017).

I. Chatziralli et al., Dexamethasone Intravitreal Implant in Diabetic Macular Edema: Real-Life Data from a Prospective Study and Predictive Factors for Visual Outcome. Diabetes therapy: research, treatment and education of diabetes and related disorders 8, 1393-1404 (2017).

I. P. Chatziralli, T. N. Sergentanis, S. Sivaprasad, HYPERREFLECTIVE FOCI AS AN INDEPENDENT VISUAL OUTCOME PREDICTOR IN MACULAR EDEMA DUE TO RETINAL VASCULAR DISEASES TREATED WITH INTRAVITREAL DEXAMETHASONE OR RANIBIZUMAB. Retina (Philadelphia, Pa.) 36, 2319-2328 (2016).

C. Framme, P. Schweizer, M. Imesch, S. Wolf, U. Wolf-Schnurrbusch, Behavior of SD-OCT-detected hyperreflective foci in the retina of anti-VEGF-treated patients with diabetic macular edema. Investigative ophthalmology & visual science 53, 5814-5818 (2012).

D. Zur et al., OCT Biomarkers as Functional Outcome Predictors in Diabetic Macular Edema Treated with Dexamethasone Implant. Ophthalmology 125, 267-275 (2018).

B. Mo, H. Y. Zhou, X. Jiao, F. Zhang, Evaluation of hyperreflective foci as a prognostic factor of visual outcome in retinal vein occlusion. International journal of ophthalmology 10, 605-612 (2017).

M. Abdel-Kader, W. M. El-Dessouky, Multifocal electroretinogram in retinal vein occlusion. Saudi journal of ophthalmology : official journal of the Saudi Ophthalmological Society 24, 125-132 (2010).

I. Audo, A. G. Robson, G. E. Holder, A. T. Moore, The negative ERG: clinical phenotypes and disease mechanisms of inner retinal dysfunction. Survey of ophthalmology 53, 16-40 (2008).

S. Yasuda et al., Significant correlation between electroretinogram parameters and ocular vascular endothelial growth factor concentration in central retinal vein occlusion eyes. Investigative ophthalmology & visual science 52, 5737-5742 (2011).

Y. Matsui, O. Katsumi, M. C. Mehta, T. Hirose, Correlation of electroretinographic and fluorescein angiographic findings in unilateral central retinal vein obstruction. Graefe's archive for clinical and experimental ophthalmology=Albrecht von Graefes Archiv fur klinische and experimentelle Ophthalmologie 232, 449-457 (1994).

H. Noma et al., Association of electroretinogram and morphological findings in branch retinal vein occlusion with macular edema. Documenta ophthalmologica. Advances in ophthalmology 123, 83-91 (2011).

H. Noma, T. Mimura, M. Kuse, K. Shimada, Association of electroretinogram and morphological findings in central retinal vein occlusion with macular edema. Clinical ophthalmology (Auckland, N. Z.) 8, 191-197 (2014).

M. E. Pitulescu, I. Schmidt, R. Benedito, R. H. Adams, Inducible gene targeting in the neonatal vasculature and analysis of retinal angiogenesis in mice. Nature protocols 5, 1518-1534 (2010).

D. J. Simon et al., A caspase cascade regulating developmental axon degeneration. The Journal of neuroscience : the official journal of the Society for Neuroscience 32, 17540-17553 (2012).

M. D. Muzumdar, B. Tasic, K. Miyamichi, L. Li, L. Luo, A global double-fluorescent Cre reporter mouse. Genesis (New York, N.Y.: 2000) 45, 593-605 (2007).

P. M. D'Onofrio, P. D. Koeberle, What can we learn about stroke from retinal ischemia models? Acta pharmacologica Sinica 34, 91-103 (2013).

P. Ramos-Cabrer, D. Padro, MRI in the Study of Animal Models of Stroke. Methods Mol Biol 1718, 377-392 (2018).

D. W. Dickson, Neuropathology of Parkinson disease. Parkinsonism Relat Disord 46 Suppl 1, S30-S33 (2018).

Y. Iturria-Medina et al., Early role of vascular dysregulation on late-onset Alzheimer's disease based on multifactorial data-driven analysis. Nature communications 7, 11934 (2016).

C. Y. Lin et al., Neurovascular abnormalities in humans and mice with Huntington's disease. Exp Neurol 250, 20-30 (2013).

S. Sarkar et al., Neurovascular changes in acute, sub-acute and chronic mouse models of Parkinson's disease. Curr Neurovasc Res 11, 48-61 (2014).

M. M. Tenreiro, R. Ferreira, L. Bernardino, M. A. Brito, Cellular response of the blood-brain barrier to injury: Potential biomarkers and therapeutic targets for brain regeneration. Neurobiol Dis 91, 262-273 (2016).

E. Zenaro, G. Piacentino, G. Constantin, The blood-brain barrier in Alzheimer's disease. Neurobiol Dis 107, 41-56 (2017).

I. Bulatovic et al., Sublethal caspase activation promotes generation of cardiomyocytes from embryonic stem cells. PloS one 10, e0120176 (2015).

T. V. Murray et al., A non-apoptotic role for caspase-9 in muscle differentiation. Journal of cell science 121, 3786-3793 (2008).

E. P. Lu et al., Caspase-9 is required for normal hematopoietic development and protection from alkylator-induced DNA damage in mice. Blood 124, 3887-3895 (2014).

H. T. Tran et al., Caspase-9 has a nonapoptotic function in Xenopus embryonic primitive blood formation. Journal of cell science 130, 2371-2381 (2017).

S. Ohsawa et al., Maturation of the olfactory sensory neurons by Apaf-1/caspase-9-mediated caspase activity. Proceedings of the National Academy of Sciences of the United States of America 107, 13366-13371 (2010).

Z. Gu et al., Skilled Movements Require Non-apoptotic Bax/Bak Pathway-Mediated Corticospinal Circuit Reorganization. Neuron 94, 626-641.e624 (2017).

N. T. Vu et al., Caspase-9b Interacts Directly with cIAP1 to Drive Agonist-Independent Activation of NF-kappaB and Lung Tumorigenesis. Cancer research 76, 2977-2989 (2016).

F. Li et al., Apoptotic cells activate the “phoenix rising” pathway to promote wound healing and tissue regeneration. Science signaling 3, ra13 (2010).

T. Wecker et al., Five-year visual acuity outcomes and injection patterns in patients with pro-re-nata treatments for AMD, DME, RVO and myopic CNV. The British journal of ophthalmology 101, 353-359 (2017).

T. Hasegawa et al., Optical coherence tomographic predictor of retinal non-perfused areas in eyes with macular oedema associated with retinal vein occlusion. The British journal of ophthalmology 101, 569-573 (2017).

C. Hernandez, M. Dal Monte, R. Simo, G. Casini, Neuroprotection as a Therapeutic Target for Diabetic Retinopathy. Journal of diabetes research 2016, 9508541 (2016).

S. Sivaprasad, S. Oyetunde, Impact of injection therapy on retinal patients with diabetic macular edema or retinal vein occlusion. Clinical ophthalmology (Auckland, N.Z.) 10, 939-946 (2016).

Y. C. Kim, B. Chiang, X. Wu, M. R. Prausnitz, Ocular delivery of macromolecules. Journal of controlled release: official journal of the Controlled Release Society 190, 172-181 (2014).

R. Gaudana, H. K. Ananthula, A. Parenky, A. K. Mitra, Ocular drug delivery. The AAPS journal 12, 348-360 (2010).

Y. Chu et al., Topical ocular delivery to laser-induced choroidal neovascularization by dual internalizing RGD and TAT peptide-modified nanoparticles. International journal of nanomedicine 12, 1353-1368 (2017).

F. de Cogan et al., Topical Delivery of Anti-VEGF Drugs to the Ocular Posterior Segment Using Cell-Penetrating Peptides. Investigative ophthalmology & visual science 58, 2578-2590 (2017).

C. Liu et al., Penetratin, a potentially powerful absorption enhancer for noninvasive intraocular drug delivery. Molecular pharmaceutics 11, 1218-1227 (2014).

K. Gaengel et al., The sphingosine-1-phosphate receptor S1PR1 restricts sprouting angiogenesis by regulating the interplay between VE-cadherin and VEGFR2. Developmental cell 23, 587-599 (2012).

C. Sun et al., NMR structure and mutagenesis of the third Bir domain of the inhibitor of apoptosis protein XIAP. The Journal of biological chemistry 275, 33777-33781 (2000).

1. A method of preventing or decreasing inflammation in a patient comprising administering to the patient in need thereof an effective amount of a caspase-9 signaling pathway inhibitor.

2. The method of claim 1, wherein the inflammation comprises neuroinflammation, appendicitis, bronchitis, bursitis, colitis, cystitis, dermatitis, encephalitis, gingivitis, meningitis, myelitis, nephritis, neuritis, periodontitis, pharyngitis, phlebitis, prostatitis, pulmonitis, retinitis, rhinitis, sinusitis, tendonitis, tonsillitis, urethritis, vaginitis, vasculitis, arthritis, myositis, arteritis, hepatitis, diverticulitis, otitis, uveitis, conjunctivitis, or episcleritis.

3. The method of claim 2, wherein the neuroinflammation comprises inflammation of a retina or a brain tissue.

4. The method of claim 3, wherein the neuroinflammation comprises inflammation of the retina characterized by retinal hyperreflective foci (HRF) and the effective amount of the caspase-9 signaling pathway inhibitor reduces the number of HRF in the patient.

5. The method of claim 1, wherein the caspase-9 signaling pathway inhibitor comprises a peptide caspase-9 inhibitor, a caspase-7 inhibitor or an Apaf-1 inhibitor.

6. The method of claim 5, wherein the peptide caspase-9 inhibitor comprises XBIR3.

7. The method of claim 1, wherein the caspase-9 signaling pathway inhibitor is conjugated to a cell-penetrating peptide.

8. The method of claim 7, wherein the cell-penetrating peptide is selected from the group consisting of Penetratin1, transportan, pIS1, Tat(48-60), pVEC, MAP, MTS, a polyarginine, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

9. The method of claim 1, wherein the caspase-9 signaling pathway inhibitor comprises XBIR3 conjugated to Penetratin1.

10. The method of claim 3, wherein the inflammation of the retina is associated with retinal vein occlusion, diabetic macular edema, retinal detachment, ocular trauma, retinitis pigmentosa, age-related macular degeneration, uveitis, a retinal degenerative disease, glaucoma, Multiple sclerosis, Behcets, Lupus, Systemic sarcoidosis, Central serous chorioretinopathy, Leber's Hereditary Optic Neuropathy, Leigh Syndrome, Stargardt, retinitis pigmentosa, Best disease, or birdshot retinopathy.

11. The method of claim 1, wherein the administering is via injection, inhalation, or topical administration.

12. The method of claim 1, wherein the patient is a human.

13. A pharmaceutical composition comprising an amount of a caspase-9 signaling pathway inhibitor effective to prevent or decrease inflammation in a patient, and a pharmaceutically acceptable carrier, wherein the pharmaceutical composition is formulated for administration to a patient via injection, inhalation, or topical administration.

14. The composition of claim 13, wherein the caspase-9 signaling pathway inhibitor comprises a peptide caspase-9 inhibitor, a caspase-7 inhibitor or an Apaf-1 inhibitor.

15. The composition of claim 14, wherein the peptide caspase-9 inhibitor comprises XBIR3.

16. The composition of claim 13, wherein the caspase-9 signaling pathway inhibitor is conjugated to a cell-penetrating peptide.

17. The composition of claim 16, wherein the cell-penetrating peptide is selected from the group consisting of Penetratin1, , transportan, pIS1, Tat(48-60), pVEC, MAP, MTS, a polyarginine, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

18. The composition of claim 13, wherein the caspase-9 signaling pathway inhibitor comprises XBIR3 conjugated to Penetratin1.

19. The composition of claim 13, wherein the effective amount decreases or prevents inflammation of the retina associated with retinal vein occlusion, diabetic macular edema, retinal detachment, ocular trauma, retinitis pigmentosa, age-related macular degeneration, uveitis, a retinal degenerative disease, glaucoma, Multiple sclerosis, Behcets, Lupus, Systemic sarcoidosis, Central serous chorioretinopathy, Leber's Hereditary Optic Neuropathy, Leigh Syndrome, Stargardt, retinitis pigmentosa, Best disease, or birdshot retinopathy.

20. The composition of claim 13, wherein the patient is a human.


SEQUENCE LISTING (50 sequences)

SEQ ID NO: 1 - protein, 118 aa, Artificial Sequence
  Description of Artificial Sequence: Synthetic polypeptide
MGSSHHHHHHSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 2 - protein, 117 aa, Artificial Sequence
  Description of Artificial Sequence: Synthetic polypeptide
MGSSHHHHHHSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 3 - protein, 17 aa, Artificial Sequence
  Description of Artificial Sequence: Synthetic peptide
  MOD_RES (1)..(1): Cys(NPys)
CRQIKIWFQNRRMKWKK

SEQ ID NO: 4 - protein, 16 aa, Artificial Sequence
  Description of Artificial Sequence: Synthetic peptide
RRLRRLLRRLLRRLRR

SEQ ID NO: 5 - protein, 12 aa, Rabies virus
RVGRRRRRRRRR

SEQ ID NO: 6 - protein, 27 aa, Artificial Sequence
  Description of Artificial Sequence: Synthetic peptide
GWTLNSAGYLLGKINLKALAALAKKIL

SEQ ID NO: 7 - protein, 16 aa, Unknown
  Description of Unknown: pIsl sequence
PVIRVWFQNKRCKDKK

SEQ ID NO: 8 - protein, 13 aa, Human immunodeficiency virus
GRKKRRQRRRPPQ

SEQ ID NO: 9 - protein, 16 aa, Mus sp.
LLIILRRRIRKQAHAH

SEQ ID NO: 10 - protein, 27 aa, Unknown
  Description of Unknown: MPS sequence
GALFLGWLGAAGSTMGAWSQPKKKRKV

SEQ ID NO: 11 - protein, 18 aa, Artificial Sequence
  Description of Artificial Sequence: Synthetic peptide
KLALKLALKALKAALKLA

SEQ ID NO: 12-13 - skipped (2 entries)

SEQ ID NO: 14 - protein, 95 aa, Unknown
  Description of Unknown: XBIR3 sequence
STNLPRNPSMADYEARIFTFGTWIYSVNKEQLARAGFYALGEGDKVKCFHCGGGLTDWKPSEDPWEQHAKWYPGCKYLLEQKGQEYINNIHLTHS

SEQ ID NO: 15 - protein, 6 aa, Artificial Sequence
  Description of Artificial Sequence: Synthetic 6xHis tag
HHHHHH

SEQ ID NO: 16 - protein, 68 aa, Homo sapiens
EEARLKSFQNWPDYAHLTPRELASAGLYYTGIGDQVQCFCCGGKLKNWEPCDRAWSEHRRHFPNCFFV

SEQ ID NO: 17 - protein, 19 aa, Artificial Sequence
  Description of Artificial Sequence: Synthetic peptide
VKRGLKLRHVRPRVTRMDV

SEQ ID NO: 18 - protein, 22 aa, Unknown
  Description of Unknown: M198 sequence
MVTVLFRRLRIRRACGPPRVRV

SEQ ID NO: 19 - protein, 22 aa, Unknown
  Description of Unknown: M1073 sequence
MVRRFLVTLRIRRACGPPRVRV

SEQ ID NO: 20 - protein, 28 aa, Bos sp.
MVKSKIGSWILVLFVAMWSDVGLCKKRP

SEQ ID NO: 21 - protein, 27 aa, Artificial Sequence
  Description of Artificial Sequence: Synthetic peptide
GALFLGFLGAAGSTMGAWSQPKKKRKV

SEQ ID NO: 22 - protein, 6 aa, Simian virus 40
KKKRKV

SEQ ID NO: 23 - protein, 17 aa, Human immunodeficiency virus
GALFLGFLGAAGSTMGA

SEQ ID NO: 24 - protein, 21 aa, Artificial Sequence
  Description of Artificial Sequence: Synthetic peptide
KETWWETWWTEWSQPKKKRKV

SEQ ID NO: 25 - protein, 12 aa, Artificial Sequence
  Description of Artificial Sequence: Synthetic peptide
KETWWETWWTEW

SEQ ID NO: 26 - protein, 18 aa, Artificial Sequence
  Description of Artificial Sequence: Synthetic peptide
LKTLTETLKELTKTLTEL

SEQ ID NO: 27 - protein, 18 aa, Artificial Sequence
  Description of Artificial Sequence: Synthetic peptide
QLALQLALQALQAALQLA

SEQ ID NO: 28 - protein, 30 aa, Artificial Sequence
  Description of Artificial Sequence: Synthetic polypeptide
WEAALAEALAEALAEHLAEALAEALEALAA

SEQ ID NO: 29 - protein, 28 aa, Unknown
  Description of Unknown: p28 sequence
LSTAADMQGVVTDGMASGLDKDYLKPDD

SEQ ID NO: 30 - protein, 12 aa, Hepatitis B virus
PLSSIFSRIGDP

SEQ ID NO: 31 - protein, 26 aa, Artificial Sequence
  Description of Artificial Sequence: Synthetic peptide
DPKGDPKGVTVTVTVTVTGKGDPKPD

SEQ ID NO: 32 - protein, 24 aa, Unknown
  Description of Unknown: bactenecin-7 sequence
RRIRPRPPRLPRPRPRPLPFPRPG

SEQ ID NO: 33 - protein, 18 aa, Unknown
  Description of Unknown: SAP sequence
VRLPPPVRLPPPVRLPPP

SEQ ID NO: 34 - protein, 18 aa, Artificial Sequence
  Description of Artificial Sequence: Synthetic peptide
VELPPPVELPPPVELPPP

SEQ ID NO: 35 - protein, 10 aa, Crotalus durissus
CRWRWKCCKK

SEQ ID NO: 36 - protein, 20 aa, Herpes simplex virus 1
HGLASTLTRWAHYNALIRAF

SEQ ID NO: 37 - protein, 10 aa, Unknown
  Description of Unknown: FGF12 sequence
PIEVCMYREP

SEQ ID NO: 38 - protein, 17 aa, Artificial Sequence
  Description of Artificial Sequence: Synthetic peptide
CSIPPEVKFNKPFVYLI

SEQ ID NO: 39 - protein, 15 aa, Artificial Sequence
  Description of Artificial Sequence: Synthetic peptide
SDLWEMMMVSLACQY

SEQ ID NO: 40 - protein, 13 aa, Artificial Sequence
  Description of Artificial Sequence: Synthetic peptide
RLSGMNEVLSFRW

SEQ ID NO: 41-42 - skipped (2 entries)

SEQ ID NO: 43 - protein, 96 aa, Unknown
  Description of Unknown: XIBR3 sequence
MSTNLPRNPSMADYEARIFTFGTWIYSVNKEQLARAGFYALGEGDKVKCFHCGGGLTDWKPSEDPWEQHAKWYPGCKYLLEQKGQEYINNIHLTHS

SEQ ID NO: 44 - protein, 69 aa, Unknown
  Description of Unknown: XIBR2 sequence
MEEARLKSFQNWPDYAHLTPRELASAGLYYTGIGDQVQCFCCGGKLKNWEPCDRAWSEHRRHFPNCFFV

SEQ ID NO: 45 - protein, 4 aa, Artificial Sequence
  Description of Artificial Sequence: Synthetic peptide
LEHD

SEQ ID NO: 46 - protein, 4 aa, Artificial Sequence
  Description of Artificial Sequence: Synthetic peptide
LEHD

SEQ ID NO: 47 - protein, 12 aa, Artificial Sequence
  Description of Artificial Sequence: Synthetic peptide
  MISC_FEATURE (1)..(12): This sequence may encompass 3-12 residues
RRRRRRRRRRRR

SEQ ID NO: 48 - protein, 18 aa, Artificial Sequence
  Description of Artificial Sequence: Synthetic peptide
  MISC_FEATURE (1)..(18): This sequence may encompass 3-6 “Pro Pro Arg” repeating units
PPRPPRPPRPPRPPRPPR

SEQ ID NO: 49 - protein, 18 aa, Artificial Sequence
  Description of Artificial Sequence: Synthetic peptide
  MISC_FEATURE (1)..(18): This sequence may encompass 3-6 “Pro Arg Arg” repeating units
PRRPRRPRRPRRPRRPRR

SEQ ID NO: 50 - protein, 8 aa, Unknown
  Description of Unknown: RING finger domain sequence
CCCHCCCC